(19) United States
(12) Patent Application Publication
(10) Pub. No.: US 2023/0080504 A1
(43) Pub. Date: Mar. 16, 2023

(54) COMPOSITE MATERIALS AND EMBOLIZATION METHODS

(71) Applicant: The Johns Hopkins University, Baltimore, MD (US)

(72) Inventors: Christos Georgiades, Baltimore, MD (US); Shin-Jae Lee, Baltimore, MD (US); Iyer Siddharth, Baltimore, MD (US); Mathias Insley, Baltimore, MD (US); Xingyu Jasmine Hu, Baltimore, MD (US); Da In Diane Lee, Baltimore, MD (US); Hai-Quan Mao, Baltimore, MD (US)

(21) Appl. No.: 17/793,207

(22) PCT Filed: Jan. 15, 2021
(86) PCT No.: PCT/US2021/013698

§ 371 Date: Jul. 15, 2022

Related U.S. Application Data
(60) Provisional application No. 62/962,177, filed on Jan. 16, 2020.

(51) Int. Cl. A61L 24/00; A61L 24/06

(52) U.S. Cl. CPC A61L 24/0036; A61L 24/0031; A61L 24/06; A61L 24/0015; A61L 2430/36; A61L 2400/06

(57) ABSTRACT

Embolization compositions and methods for controlling undesired bleeding and other treatments are provided. Preferred composition may comprise (a) a crosslinked hydrogel material; and (b) a fiber material, wherein the composition comprises a plurality of macropores; and the hydrogel material and fiber material are bonded by covalent and/or non-covalent bonds.

The present application claims the benefit of U.S. provisional application No. 62/962,177 filed Jan. 16, 2020, which is incorporated herein by reference in its entirety.

### BACKGROUND

### 1. Field

The present disclosure relates to embolization compositions and methods for controlling undesired bleeding.

### 2. Background

Transcatheter embolization is becoming an increasingly common clinical procedure, due to the growing number of clinical indications that can be treated with this minimally invasive approach, as well as an aging population. Current approaches include use of platinum coils, which induce thrombus formation to block blood flow. However, platinum coils are highly vessel specific, cause imaging artifacts in CT scans, are high in cost, and require significant skill to deliver in a sufficiently compacted state to prevent eventual recanalization.

It thus would be desirable to have new materials and methods for embolization therapies.

### SUMMARY

We now provide new composition that can be particularly suitable for use in embolization applications.

In a first aspect, an embolization composition is provided that comprises: (a) a crosslinked hydrogel material; and (b) a fiber material.

Preferred compositions are macroporous and thus may contain a distribution or pores that have a pore size of at least about 10 microns such at least about 20, 30, 40, 50, 60, 70, 80 90 or 100 microns. Preferred macropore sizes may be less about 500, 400, 300, 200, 150, 120 or 100 microns.

Preferred embolization compositions also are catheter-compatible and can be delivered to a subject through a catheter.

In preferred embolization compositions, the hydrogel material and the fiber material will share covalent and/or non-covalent bonds. Thus, for instance, the hydrogel material and fiber material may be bonded by one or more electron sharing bonds such as one or more of an ionic bond, hydrogen bond or a covalent bond.

In certain aspects, a composition contains fiber material covalently bonded to hydrogel material. In this aspect, the embolization composition includes a cross-linking moiety present in an amount effective to induce cross-linking between fiber material and hydrogel material.

The covalent linkage may include a variety of reacted moieties, including for instance a reacted acrylate that may be present on either or both fiber material or hydrogel material prior to covalent bond formation, or other reacted moieties such as acrylamide, reacted and/or a vinyl ether moiety.

In certain embodiments, fiber material and hydrogel material may have minimal or no crosslinking, such as where less 30, 20, 10 5, 4, 3, 2, 1, 0.5 or 0.1 percent of reactive groups such as acrylate present on hydrogen and/or fiber materials are reacted as determined by NMR or other analysis, or where hydrogen and/or fiber materials comprise minimal or no functional groups for crosslinking.

In certain preferred compositions, fiber material and hydrogel material also may share ionic and/or hydrogen bonds, for example where the hydrogel material and/or the fiber material comprise functional groups that can form ionic bonds between the hydrogel and fiber material. For instance, the hydrogel material and/or fiber material may comprise polar moieties such as hydroxy, carboxy, cyano and/or nitro groups which can form ionic or hydrogen bonds.

In preferred embolization compositions, the hydrogel material and the fiber material will share both covalent and non-covalent bonds. For example, the hydrogel material and the fiber material may be both 1) covalently linked and 2) share ionic bonds.

In particular aspects, the hydrogel material and fiber material of a composition are a cryogel composite (i.e. produced through a cryogelation process) and macropores are produced through the cryogel process.

In one aspect, the fiber material includes a non-woven polymeric fiber. In certain aspects, the polymeric fiber includes an electrospun polycaprolactone fiber. In additional aspects, the fiber material may include a synthetic polymeric material comprising a poly(lactic-co-glycolic acid), a polylactic acid, and/or a polycaprolactone, or other material such as silk, collagen, elastin, hyaluronic acid, chitosan, or a combination thereof.

In an additional embodiment, the polymeric fiber includes a biocompatible biodegradable polyester.

In certain aspects, the fiber material preferably may be an electrospun fiber. Fiber materials also may be plasma-treated fibers, for example to produce functional groups for covalent or non-covalent bonding with hydrogel material.

The fiber material suitably may have varying dimensions. For instance, preferred materials include polymeric fibers having a mean diameter of from about 100 nm to 8000 nm, and a longest dimension of 500 micrometers or less.

The weight ratio of fiber material to hydrogel material in a composition may suitably vary and may be for example from about 1:100 to about 10:1, more typically from about 1:10 to about 10:1.

In one preferred embodiment, the hydrogel material includes an alginate material. In certain aspects, the hydrogel material includes a poly(ethylene glycol), a collagen, an alginate, a dextran, an elastin, a fibrin, a hyaluronic acid, a poly(vinyl alcohol), or a combination thereof.

As discussed, the embolization composition comprises includes a plurality of macropores present on or within a surface of the composition, where the macropores suitably are present at a concentration of at least about 50 pores per cm² of the composition surface, and where at least 80% of the pores have an average pore diameter on the surface is at least about 10 microns, and more preferably where at least 80% of the pores have an average pore diameter on the surface is at least about 15, 20, 30, 40, 50 microns but with the average pore size less than 300, 200, 120 or 100 microns.

In a preferred aspect, an embolization composition may also comprise a thermoresponsive material which suitably may be present as a separate composition component (i.e., not covalently linked to another component, particularly hydrogel or fiber material), or the thermoresponsive material may be incorporated with another composition such as fiber or hydrogel material. In one embodiment, the thermoresponsive material may include one or more of poly(N-isopropylacrylamide) (PNIPAM), poly[2-(dimethylamino)ethyl methacrylate] (pDMAEMA), hydroxypropylcellulose, poly(vinylcaprolactame), polyethylene oxide, polyvinylmethylether, polyhydroxyethylmethacrylate and polyvinyl methyl ether. In one embodiment, the thermoresponsive material is PNIPAM. Preferably, the thermoresponsive material is covalently linked to hydrogel material or fiber material components of a composition.

In preferred systems, at elevated temperatures such as body temperatures present when an embolization composition is administered to a subject, the thermoresponsive material may contract and thereby favorably increase stiffening of an embolization composition containing the thermoresponsive material.

In a further preferred aspect, an embolization composition may also comprise a detetable material such as a contrast agent or radiopaque label that can allow for detection of the embolization composition after administration to a subject. For instance, a barium, tantalum, tungsten or bismuth marker may be incorporated into a composition such as by reaction of a hydrogel or fiber material with appropriate agents, for example barium sulfate, bismuth trioxide, or tantalum oxide.

In a yet further preferred aspect, an embolization composition may also comprise a gelatin, collagen, or fibrin material in addition to the hydrogel material and fiber material.

In a preferred aspect, an embolization composition is provided that has regions of differing relative stiffness through the volume of the embolization composition. For instance, a particular embolization sample may have 2, 3, 4, 5, 6, 7, 8, 9, 10 or more regions of differing stiffness at room temperature (e.g. 25° C.) and/or physiological temperatures (37° C.). In certain embolization compositions having such regions of differing stiffness, the stiffness of a region may suitably differ from the stiffness of an adjacent region by a Young's modulus (E) of at least 0.25 kPa, 0.5 kPa, 1.0 kPa, 2.0 kPa, 3.0 kPa, 4.0 kPa, 5.0 kPa, 6.0 kPa, 7.0 kPa, or 8.0 kPa. Such multi-region embolization compositions may be suitably prepared by a multi-step cryogel process with differing fiber loadings, or use of other stiffening materials, as further discussed below.

In further particular aspects, an embolization composition may comprise a non-hydrogel material in addition to a crosslinked hydrogel material and a fiber material.

In additional aspects, compositions are provided that can exhibit enhanced “foldability” i.e. where a greater volume of the embolization composition can be effectively advanced through a catheter of a specific diameter. With such compositions, a composition sample can be advanced such as with guidewire or other instrument that applies pressure in the center region of the sample through a catheter with sample regions offset from the center area fold back from the center to facilitate passage through the catheter and consequently enhance the volume of the embolization composition that can be advanced through the catheter. Such enhanced foldability properties can be enhanced by providing interposed regions of differing stiffness along a dimension of the embolization material. For example, a composition region of relative enhanced stiffness may be bordered by regions of reduced stiffness. For instance, such interposing regions of reduced stiffness may suitably have a stiffness that differs from the stiffness of an adjacent region by a Young's modulus (E) of at least 0.25 kPa, 0.5 kPa, 1.0 kPa, 2.0 kPa, 3.0 kPa, 4.0 kPa, 5.0 kPa, 6.0 kPa, 7.0 kPa, or 8.0 kPa.

Such enhanced foldability properties also may be provided by selected patterning and layering to provide a composition with the desired folding characteristics. See, for instance, the procedures set forth in Examples 4 and 5 which follow.

In certain preferred systems, regions of differing stiffness or distinct patterning in a composition sample may be at least substantially symmetric within a composition sample. For instance, each distinct region may comprise the same volume (or within 1, 5, 8, 10, 12, 15 or 20 volume percent of each other) as one, two or more or all other regions of the composition samples based on total volume of the composition sample.

In other preferred systems, regions of differing stiffness or distinct patterning in a composition sample may be asymmetric within a sample. For instance, each distinct region may comprise a differing volume (e.g. at least 5, 8, 10, 15, 20, 25, 30, 40, 50, 60, 70, 80 volume percent difference between distinct regions including adjacent regions) relative to one, two or more or all other regions of the composition samples based on total volume of the composition sample.

In preferred systems, a composition may comprise one or more therapeutic agents, including for example one or more anti-cancer agents and/or one or more thrombogenic agents. Such therapeutic agents may be a component of a hydrogel or fiber component (e.g. where the therapeutic agent is covalently linked to a hydrogel or fiber material), or where a therapeutic agent is a separate (i.e. not covalently linked to another composition component) material of the composition.

In preferred systems, a composition may comprise one or more thrombogenic agents on surface layer of the device with direct contact with blood. In one aspect, such thrombogenic agents may be a component of a hydrogel or fiber component (e.g. where the therapeutic agent is covalently linked to a hydrogel or fiber material). In another aspect, the one or more thrombogenic agents may be a separate (i.e. not covalently linked to another composition component) material of the composition.

In particular aspects, a composition may comprise a therapeutic agent that is present in differing concentrations in different composition regions. For instance, in one preferred system, a composition may be formed by patterning as exemplified in Examples 4 and 5 which follow where one or more therapeutic agents are present in the highest concentration in the outermost composition region to provide a more rapid and controlled dosage of the therapeutic agent to a patient. An outermost composition region as referred to herein would i) interface another composition region on a first outermost region side and ii) be the exposed surface of the composition sample on a second outermost region side. In another preferred system, one or more therapeutic agents may be loaded in an inner composition region to provide a delayed administration of the loaded therapeutic agent to a patient, including to coordinate sequential dosing with therapeutic agent(s) loaded in other composition regions, such as an outermost region.

In particular aspects, for a composition that comprises one or more therapeutic agents that are present in differing concentrations in different composition regions suitably about 10 to 90, or 30 to 90, or 50 to 90 weight percent of the total therapeutics agent(s) present in an administered composition may be present in a single region of the composition such as the outermost region. A single region of the composition typically may be up to or at least 10, 20, 30, 40, 50, 60, 70, 80 or 90 percent of the total volume of the administered composition sample.

In certain preferred systems, concentrations of therapeutic agent(s) present in various regions of a composition sample may be asymmetric within a sample. For instance, each distinct region may comprise a differing weight amount of therapeutic agent(s) (e.g. at least 3, 5, 8, 10, 15, 20, 25, 30, 40, 50, 60, 70, 80 weight percent difference of therapeutic agent(s) between distinct regions including adjacent regions) relative to one, two or more or all other regions of the composition samples. In this aspect, a composition region may be defined having a weight amount of therapeutic agents that differ by at least 3, 5, 10, 15, 20, 30, 40 or 50 weight percent relative to weight amount of the same therapeutic agent(s) in an adjacent distinct region.

In certain preferred systems, concentrations of therapeutic agent(s) present in adjacent regions of a composition sample may be more symmetric within a sample. For instance, each distinct region may comprise a weight amount (e.g. within about 10, 9, 8, 7, 6, 5, 4, 3, 2 or 1 at least 3, 5, 8, 10, 15, 20, 25, 30, 40, 50, 60, 70, 80 weight percent difference of therapeutic agent(s) between distinct regions including adjacent regionsr) relative to one, two or more or all other regions of the composition samples.

As discussed, in certain systems, a composition may be substantially symmetric (including radial symmetry) with respect to patterning or therapeutic agent(s) concentration. Such symmetry may be with respect to spatial configuration and arrangement of the composition and regions thereof.

In certain other systems, a composition may be asymmetric (including radial asymmetry) with respect to patterning or therapeutic agent(s) concentration. Such asymmetry may be with respect to spatial configuration and arrangement of the composition and regions thereof. Asymmetry of the composition also may be along the along the length of an applied composition or device (e.g. along the length of the blood vessel) which may provide favorable properties including in view of blood flow direction.

In certain embodiments, an embolization composition is formulated for administration to a subject by injection. In other embodiments, an embolization composition is formulated for subdermal administration.

Methods for embolizing a blood vessel of a subject such as a mammal particularly a human are also provided which suitably may comprise delivering via a catheter into the blood vessel an embolization composition as disclosed herein.

In certain aspects, embolization compositions are particularly useful in treatment of cancer, including solid tumors. In such treatments, the embolization composition may be delivered at the tumor site for example via transcatheter delivery of the embolization composition. One or more anti-cancer agents may be incorporated into the embolization composition for delivery to the tumor.

Treatment kits are also provided that suitably comprise an embolization composition as disclosed herein and optionally a medical device, particularly an administration device such as a catheter. A kit also optionally may include instructions, particularly written instructions such as a package insert or label, for use of the composition and kit.

Unless otherwise specified herein, references to an “embolization” composition are not limiting to embolization and for example such compositions can be employed for applications other than embolization. For instance, a composition disclosed herein (which may be referred to herein as am embolization composition) may be used (including primary use) as a composition to deliver one or more therapeutic agents.

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic of the synthesis procedure for macroporous composites with interfacial bonding.

FIG. 2 is a schematic of thermoresponsive composite with interfacial bonding.

FIG. 3 is a schematic of alginate purification and acrylation (methacrylation shown).

FIG. 4 Control of methacrylation degree based on AEMA-to-alginate mass ratio during carbodiimide functionalization reaction

FIG. 5 Images of alginate-PCL composite gels formed at −20° C. (left) and room temperature (right).

FIG. 6 shows rheological properties of 20% and 30% methacrylated alginate cryogels with and without ionic crosslinking.

FIG. 7 shows the effect of dual crosslinking on G′.

FIG. 8 shows MA-PCL fiber preparation.

FIG. 9 shows a TBO assay for plasma treatment and methacrylation efficiency.

FIG. 10 depicts graphically enhanced composite stiffening through interfacial bonding.

FIG. 11 depicts composite stiffening with functionalized PCL.

FIG. 12 depicts graphically increased elasticity with higher MA-PCL loading.

FIG. 13 shows injectability heat maps.

FIG. 14 shows injectability scoring criteria. Score increases from 1 to 4 going left to right. A score of 0 is given if the gel cannot pass through the needle.

FIG. 15 shows iterative compression curves.

FIG. 16 (includes FIGS. 16A-16H) shows SEM micrographs. (A) 1% alginate, (B) 1.5% alginate, (C) 2% alginate, (D) 1% non-macroporous (note scale bar), (E) 1% alginate+20 mg/mL MA-PCL, (F), 1% alginate+30 mg/mL, (G) 1% alginate+20 mg/mL (higher magnification), (H) 1% alginate+30 mg/mL (higher magnification).

FIG. 17 shows X-ray of barium-loaded cryogels. From left to right, the Ba′ concentrations are 0 mM, 10 mM, 25 mM, and 50 mM.

FIG. 18 shows NIPAM nanogel size based on surfactant concentration during polymerization.

FIG. 19 shows DLS intensity of MA-NNGs at RT (˜500 nm) and 37° C. (˜100 nm)

FIG. 20 shows a summary of thermoresponsive properties of various embolizartion compositons.

FIG. 21 shows representative temperature sweeps curves of 1% alginate with 1:1 Alginate:NIPAM mass ratio.

FIG. 22 shows fiber sintering effect on G′.

FIG. 23 shows SEM micrographs of 1% alginate+30 mg/mL MA-PCL before (left) and after (right) heating for 10 minutes at 57° C.

FIGS. 24A-D shows results of Example 4 which follows.

FIGS. 25A-G shows of Example 5 which follows.

FIG. 26 shows needle/catheter loading and injection of Example 6 which follows.

FIG. 27 shows injectability scores for 1% or 1.5% alginate, 1% fiber cryogels of Example 6 which follows.

### DETAILED DESCRIPTION

As discussed, we now provide a macroporous hydrogel-fiber composite with interfacial bonding that is particularly useful as an embolization agent. In one specifically preferred aspect, acrylated (including methacrylated) alginate is polymerized in cryogenic conditions and is subsequently crosslinked (e.g. using divalent cations), resulting in a macroporous structure. Fiber materials including preferred electrospun polymers such as a methacrylated electrospun poly(ε-caprolactone) fibers can be added to the alginate hydrogel in various concentrations to enhance stiffness and elasticity without adversely detracting from compressibility, allowing for a composite with appropriate mechanical properties to be delivered through a catheter.

Further, the resulting compositions can fill blood vessels through re-expansion, rather than re-coiling, making them less specific to vessel size and morphology. That is, the mode of vessel occlusion via re-expansion of the composite makes the material a versatile embolic agent, appropriate for a range of blood vessel sizes and morphologies.

In a particular aspect, a thermoresponsive component has been incorporated into the macroporous composite, which can induce greater stiffness and elasticity in situ under elevated physiological temperatures. That is, the thermoresponse has been shown to induce stiffening of the macroporous composite at physiological temperatures without a significant reduction in overall volume, which can be used to further increase efficacy of the device by causing the material to be soft during delivery to allow for increased catheter compliance, while stiffening and becoming more elastic in situ to improve embolus stability.

The fiber material also may be sintered as an additional stiffening strategy.

Thus, preferred compositions of the invention can have multiple tunable structural, swelling, and mechanical properties, and an in situ stiffening effect, that are specifically designed as a versatile alternative to platinum coils and other existing embolization products.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The polymeric matrix may be formed of any suitable synthetic or naturally occurring polymer material. As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

The term “polymer” or “fiber” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da. Polymers and oligomers may be naturally occurring or obtained from synthetic sources.

The term “acrylate” as used herein includes unsubstituted acrylates (e.g. CH₂═CH₂C(O)OR where R is a hydrogen or non-hydrogen substituent) as well as methacrylates and other substituted acrylates (e.g. CH₂═CHR′C(O)OR where R′ is a non-substituent such as optionally substituted C₁₋₆ alkyl including methyl and trifluoromethyl and R is a hydrogen or non-hydrogen substituent.

The term “catheter-compatible” as referred to herein indicates a composition is loadable and deliverable to a patient through an embolization catheter.

### Compositions

As discussed, the present compositions preferably contain a fiber material, such as a polymer generally having a mean diameter of from about 10 nm to about 10,000 nm, such as about 100 nm to about 8000 nm, or about 150 nm to about 5,000 nm, or about 100, 150, 200, 250, 300, 350, 400, 450, 500, 600, 700, 800, 900, 1,000, 1,500, 2,000, 2,500, 3,000, 3,500, 4,000, 4,500, 5,000, 5,500, 6,000, 6,500, 7,000, 7,500, or 8,000.

As provided herein, the ratio of fiber material to hydrogel material suitably can vary. For example, the ratio of polymeric fiber to hydrogel material is suitably from about 1:100 to about 100:1 on a component-mass basis, such as about 1:50 to about 50:1, or 1:10 to about 10:1, such as 1:5 to about 5:1, such as about 1:3 to about 3:1. The ratio of polymeric fiber to hydrogel material is also provided as a concentration basis, e.g., a given weight of fiber material per volume of hydrogel material. For example the concentration is from about 1 to 50 mg/mL.

As discussed, the present compositions are macroporous. Porosity can be introduced to the composite by a number of techniques, including under cryogenic conditions. See, for instance, the examples which follow for an exemplary pore-forming protocol. The presence, size, distribution, frequency and other parameters of the pores can be modulated during the synthesis of the fiber/hydrogel composite. As discussed, average pore size is preferred at least 10 microns and more preferably is at least 20, 30, 40 or 50 microns. Pore size may be narrowly tailored, e.g., such that at least 40%, such as 50%, 60%, 70%, 80%, 90%, 95% or greater than 95% of the pores are in a desired size or within a desired size range.

The hydrogel composite of the invention can include any type of suitable hydrogel component. The invention contemplate nanostructure/gel composites that include any suitable gel component, including any suitable hydrogel component known in the art. The gel and/or hydrogels can be formed of any suitable synthetic or naturally-occurring materials.

For example, the polymer component of the gels and/or hydrogels can comprise a cellulose ester, for example, cellulose acetate, cellulose acetate propionate (CAP), cellulose acetate butyrate (CAB), cellulose propionate (CP), cellulose butyrate (CB), cellulose propionate butyrate (CPB), cellulose diacetate (CDA), cellulose triacetate (CTA), or the like. These cellulose esters are described in U.S. Pat. Nos. 1,698,049, 1,683,347, 1,880,808, 1,880,560, 1,984,147, 2,129,052, and 3,617,201, and may be prepared using techniques known in the art or obtained commercially. Commercially available cellulose esters suitable herein include CA 320, CA 398, CAB 381, CAB 551, CAB 553, CAP 482, CAP 504, all available from Eastman Chemical Company, Kingsport, Tenn. Such cellulose esters typically have a number average molecular weight of between about 10,000 and about 75,000.

The cellulose esters and comprise a mixture of cellulose and cellulose ester monomer units; for example, commercially available cellulose acetate butyrate contains cellulose acetate monomer units as well as cellulose butyrate monomer units and unesterified cellulose units.

The hydrogels of the present compositions may also be comprised of other water-swellable polymers, such as acrylate polymers, which are generally formed from acrylic acid, methacrylic acid, methyl acrylate, ethyl acrylate, methyl methacrylate, ethyl methacrylate, and/or other vinyl monomers. Suitable acrylate polymers are those copolymers available under the tradename “Eudragit” from Rohm Pharma (Germany), as indicated supra. The Eudragit series E, L, S, RL, RS and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. Particularly preferred such copolymers are Eudragit L-30D-55 and Eudragit L-100-55 (the latter copolymer is a spray-dried form of Eudragit L-30D-55 that can be reconstituted with water). The molecular weight of the Eudragit L-30D-55 and Eudragit L-100-55 copolymer is approximately 135,000 Da, with a ratio of free carboxyl groups to ester groups of approximately 1:1. The copolymer is generally insoluble in aqueous fluids having a pH below 5.5. Another particularly suitable methacrylic acid-methyl methacrylate copolymer is Eudragit S-100, which differs from Eudragit L-30D-55 in that the ratio of free carboxyl groups to ester groups is approximately 1:2. Eudragit S-100 is insoluble at pH below 5.5, but unlike Eudragit L-30D-55, is poorly soluble in aqueous fluids having a pH in the range of 5.5 to 7.0. This copolymer is soluble at pH 7.0 and above. Eudragit L-100 may also be used, which has a pH-dependent solubility profile between that of Eudragit L-30D-55 and Eudragit S-100, insofar as it is insoluble at a pH below 6.0. It will be appreciated by those skilled in the art that Eudragit L-30D-55, L-100-55, L-100, and S-100 can be replaced with other acceptable polymers having similar pH-dependent solubility characteristics.

Any of the herein-described hydrogel compositions may be modified so as to contain an active agent and thereby act as an active agent delivery system when applied to a body surface (e.g., a site of tissue repair) in active agent-transmitting relation thereto. The release of active agents “loaded” into the present hydrogel compositions of the invention typically involves both absorption of water and desorption of the agent via a swelling-controlled diffusion mechanism. Active agent-containing hydrogel compositions may be employed, by way of example, in transdermal drug delivery systems, in wound dressings, in topical pharmaceutical formulations, in implanted drug delivery systems, and the like.

In various other embodiments, the composite materials of the invention can be based on hyaluronic acid (HA) as the hydrogel material. HA is a non-sulfated, linear polysaccharide with repeating disaccharide units which form the hydrogel component. HA is also a non-immunogenic, native component of the extracellular matrix in human tissues, and widely used as a dermal filler in aesthetic and reconstructive procedures.

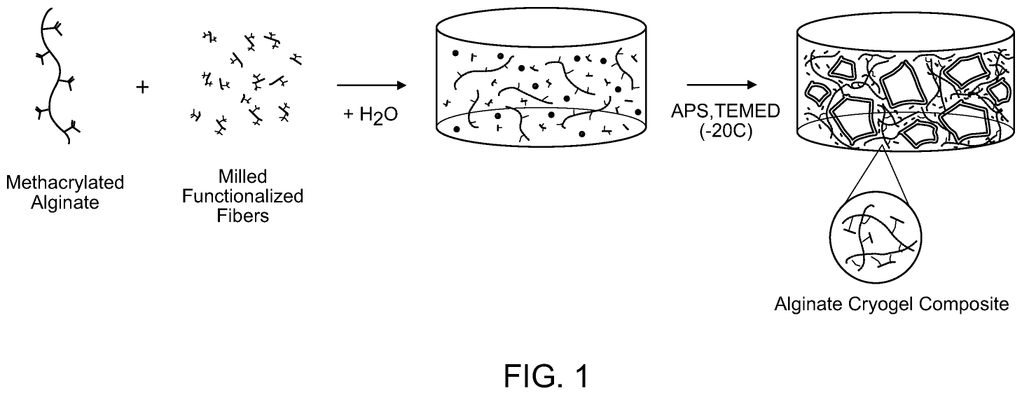

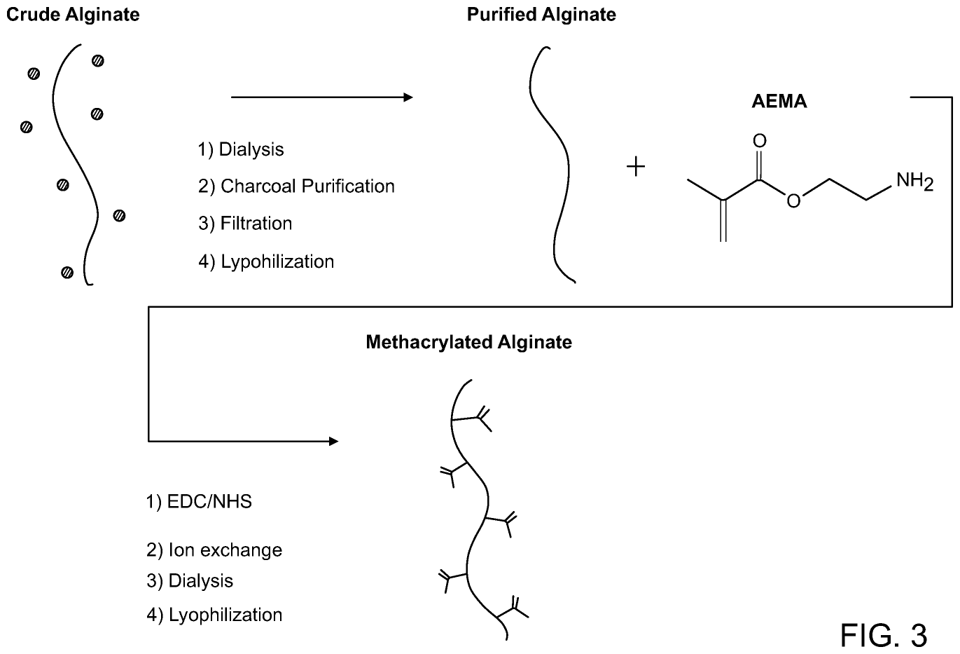

The present embolization compositions can be prepared by a variety of methods and generally include admixing the composition components. Cryogel procedures are generally preferred and can facilitate introduction of a macroporous system in the composition. FIGS. 1 and 3 depict preferred preparation methods. The examples which follow also disclose preferred methods.

Thus, FIG. 1 depicts admixing hydrogel material (such as the depicted methacrylated alginate) and fiber material (such as the depicted functionalized fibers which may be e.g. methacrylated electrospun poly(ε-caprolactone) (PCL) fibers). That mixture then undergoes cryogelation which can involve crystallization/solidification of the solvent such as water. That process can produce a system of macropores in the composite material. See FIG. 1 and the examples which follow.

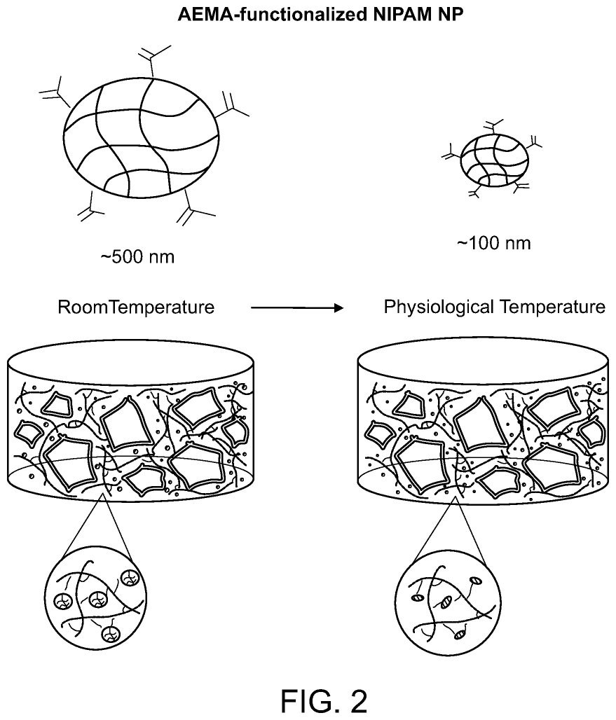

A thermoresponsive material can be introduced into the composition in a variety of ways. For instance, in one specifically preferred aspect, N-isopropylacrylamide (NIPAM), a thermoresponsive compound exhibiting a lower critical solution temperature (LCST) at ˜32° C., is co-polymerized with acrylic acid (AAc) and N,N′-Methylenebis(acrylamide) (MBA) in the presence of a surfactant to form nanogels, which are functionalized to be covalently incorporated into the alginate cryogel structure. See FIG. 2 and the examples which follow. The resulting composite exhibited increased stiffness and elasticity under physiological temperatures.

In the cryogel process, suitable amounts of fiber material and hydrogel material admixed as a precursor solution (e.g. aqueous mixture) suitably may vary. In one aspect, in a aqueous composition prepared before the cryogel process (precursor composition), 0.1 weight percent to 5, 6, 7, 8, 9, or 10 weight percent of hydrogel material may be used based on total weight of the aqueous precursor composition, and 0.5 to 10 weight percent of fiber material may be used based on total weight of the aqueous precursor composition. If a thermoresponsive agent is utilized, 0.1 weight percent to 1, 2, 3, or 5 weight percent of thermoresponsive material may be used based on total weight of the aqueous precursor composition,

To prepare embolization compositions that have regions of differing stiffness as discussed above, a multiple step cryogel process suitably may be employed. Thus, a first cryogel composition with a first fiber material loading amount (thus providing a first stiffness) may be prepared, the resulting cryogel composite may be allowed to thaw moderately and a second cryogel composition with a second, distinct fiber material loading amount (thus providing a second distinct stiffness) can be prepared over the first prepared composite. The process can be repeated to provide additional adjacent composite regions of differing stiffness based on the differing fiber loadings of each of the process stages. The partial thaw step of the previously prepared composite facilitates integration of the regions of differing stiffness to provide a more robust, unitary final composite.

### Crosslinking

As discussed, a hydrogel of the present compositions may be covalently crosslinked. Crosslinking may be desired as between the polymers of the hydrogel component, but also crosslinking may be desired as between the polymers of the hydrogel and other composition components. The invention contemplates any suitable means for crosslinking polymers to one another, and crosslinking the hydrogel polymers with other composition components. The crosslinks may be formed using any suitable means, including using heat, radiation, fre-radical initiator or other chemical curing (crosslinking) agent. The degree of crosslinking should be sufficient to eliminate or at least minimize cold flow under compression.

In certain preferred aspects, a free radical polymerization initiator is used, and can be any of the known free radical-generating initiators conventionally used in vinyl polymerization. Preferred initiators are organic peroxides and azo compounds, generally used in an amount from about 0.01 wt. % to 15 wt. %, preferably 0.05 wt. % to 10 wt. %, more preferably from about 0.1 wt. % to about 5% and most preferably from about 0.5 wt. % to about 4 wt. % of the polymerizable material. Suitable organic peroxides include dialkyl peroxides such as t-butyl peroxide and 2,2bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds include azo bis (isobutyronitrile) and azo bis (2,4-dimethylvaleronitrile). The temperature for thermally crosslinking will depend on the actual components and may be readily deduced by one of ordinary skill in the art, but typically ranges from about 80° C. to about 200° C.

Crosslinking may also be accomplished with radiation, typically in the presence of a photoinitiator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG, Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa(3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri(3-mercaptopropionate), trimethylolethane trithioglycolate, trimethylolpropane tri(3-mercaptopropionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

### Fiber Materials

Fiber materials of the present compositions suitably may be fabricated by electrostatic spinning (also referred to as electrospinning). The technique of electrospinning of liquids and/or solutions capable of forming fibers, is well known and has been described in a number of patents, such as, for example, U.S. Pat. Nos. 4,043,331 and 5,522,879. The process of electrospinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening).

The process of electrostatic spinning has typically been directed toward the use of the fibers to create a mat or other non-woven material, as disclosed, for example, in U.S. Pat. No. 4,043,331. Nanofibers ranging from 50 nm to 5 micrometers in diameter can be electrospun into a nonwoven or an aligned nanofiber mesh. Due to the small fiber diameters, electrospun textiles inherently possess a very high surface area and a small pore size. These properties make electrospun fabrics potential candidates for a number of applications including: membranes, tissue scaffolding, and other biomedical applications.

Electrostatically spun fibers can be produced having very thin diameters. Parameters that influence the diameter, consistency, and uniformity of the electrospun fibers include the polymeric material and cross-linker concentration (loading) in the fiber-forming combination, the applied voltage, and needle collector distance. According to one embodiment of the present invention, a nanofiber has a diameter ranging from about 1 nm to about 100.mu.m. In other embodiments, the nanofiber has a diameter in a range of about 1 nm to about 1000 nm. Further, the nanofiber may have an aspect ratio in a range of at least about 10 to about at least 100. It will be appreciated that, because of the very small diameter of the fibers, the fibers have a high surface area per unit of mass. This high surface area to mass ratio permits fiber-forming solutions or liquids to be transformed from liquid or solvated fiber-forming materials to solid nanofibers in fractions of a second.

A variety of fiber materials may be used in the present compositions. Depending upon the intended application, the fiber-forming polymeric material may be hydrophilic, hydrophobic or amphiphilic. Additionally, the fiber-forming polymeric material may be a thermally responsive polymeric material.

Synthetic or natural, biodegradable or non-biodegradable polymers may form the nanofibers/nanostructures of the invention. A “synthetic polymer” refers to a polymer that is synthetically prepared and that includes non-naturally occurring monomeric units. For example, a synthetic polymer can include non-natural monomeric units such as acrylate or acrylamide units. Synthetic polymers are typically formed by traditional polymerization reactions, such as addition, condensation, or free-radical polymerizations. Synthetic polymers can also include those having natural monomeric units, such as naturally-occurring peptide, nucleotide, and saccharide monomeric units in combination with non-natural monomeric units (for example synthetic peptide, nucleotide, and saccharide derivatives). These types of synthetic polymers can be produced by standard synthetic techniques, such as by solid phase synthesis, or recombinantly, when allowed.

A “natural polymer” refers to a polymer that is either naturally, recombinantly, or synthetically prepared and that consists of naturally occurring monomeric units in the polymeric backbone. In some cases, the natural polymer may be modified, processed, derivatized, or otherwise treated to change the chemical and/or physical properties of the natural polymer. In these instances, the term “natural polymer” will be modified to reflect the change to the natural polymer (for example, a “derivatized natural polymer”, or a “deglycosylated natural polymer”).

Fiber materials, for example, may include both addition polymer and condensation polymer materials such as polyolefin, polyacetal, polyamide, polyester, cellulose ether and ester, polyalkylene sulfide, polyarylene oxide, polysulfone, modified polysulfone polymers and mixtures thereof. Exemplary materials within these generic classes include polyethylene, poly(.epsilon.-caprolactone), poly(lactate), poly(glycolate), polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms. Exemplary addition polymers tend to be glassy (a Tg greater than room temperature). This is the case for polyvinylchloride and polymethylmethacrylate, polystyrene polymer compositions, or alloys or low in crystallinity for polyvinylidene fluoride and polyvinyl alcohol materials.

Block copolymers can also be used as fiber materials. In preparing a composition for the preparation of nanofibers, a solvent system can be chosen such that both blocks are soluble in the solvent. One example is an ABA (styrene-EP-styrene) or AB (styrene-EP) polymer in methylene chloride solvent. Examples of such block copolymers are a Kraton-type of AB and ABA block polymers including styrene/butadiene and styrene/hydrogenated butadiene(ethylene propylene), a Pebax-type of epsilon-caprolactam/ethylene oxide and a Sympatex-type of polyester/ethylene oxide and polyurethanes of ethylene oxide and isocyanates.

Addition polymers such as polyvinylidene fluoride, syndiotactic polystyrene, copolymers of vinylidene fluoride and hexafluoropropylene, polyvinyl alcohol, polyvinyl acetate, amorphous addition polymers, such as poly(acrylonitrile) and its copolymers with acrylic acid and methacrylates, polystyrene, poly(vinyl chloride) and its various copolymers, poly(methyl methacrylate) and its various copolymers, can be solution spun with relative ease because they are soluble at low pressures and temperatures. Highly crystalline polymer like polyethylene and polypropylene generally requires higher temperature and high vapor pressure solvents if they are to be solution spun.

Fiber materials can also be formed from polymeric compositions comprising two or more polymeric materials in polymer admixture, alloy format, or in a crosslinked chemically bonded structure. Two related polymer materials can be blended to provide the nanofiber with beneficial properties. For example, a high molecular weight polyvinylchloride can be blended with a low molecular weight polyvinylchloride. Similarly, a high molecular weight nylon material can be blended with a low molecular weight nylon material. Further, differing species of a general polymeric genus can be blended. For example, a high molecular weight styrene material can be blended with a low molecular weight, high impact polystyrene. A Nylon-6 material can be blended with a nylon copolymer such as a Nylon-6; 6,6; 6,10 copolymer. Further, a polyvinyl alcohol having a low degree of hydrolysis such as an 87% hydrolyzed polyvinyl alcohol can be blended with a fully or super hydrolyzed polyvinyl alcohol having a degree of hydrolysis between 98 and 99.9% and higher. All of these materials in admixture can be crosslinked using appropriate crosslinking mechanisms. Nylons can be crosslinked using crosslinking agents that are reactive with the nitrogen atom in the amide linkage. Polyvinyl alcohol materials can be crosslinked using hydroxyl reactive materials such as monoaldehydes, such as formaldehyde, ureas, melamine-formaldehyde resin and its analogues, boric acids, and other inorganic compounds, dialdehydes, diacids, urethanes, epoxies, and other known crosslinking agents. Crosslinking reagent reacts and forms covalent bonds between polymer chains to substantially improve molecular weight, chemical resistance, overall strength and resistance to mechanical degradation.

Biodegradable polymers can also be used in the preparation of the nanostructures of the invention. Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with, for example, implantable medical devices include polylactide, polyglycolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used.

In some embodiments, the fiber materials are non-biodegradable polymers. Non-biodegradable refers to polymers that are generally not able to be non-enzymatically, hydrolytically or enzymatically degraded. For example, the non-biodegradable polymer is resistant to degradation that may be caused by proteases. Non-biodegradable polymers may include either natural or synthetic polymers.

The inclusion of cross-linking agents within the composition forming the nanofiber, allows the nanofiber to be compatible with a wide range of support surfaces. The cross-linking agents can be used alone or in combination with other materials to provide a desired surface characteristic.

Suitable cross-linking agents include either monomeric (small molecule materials) or polymeric materials having at least two latent reactive activatable groups that are capable of forming covalent bonds with other materials when subjected to a source of energy such as radiation, electrical or thermal energy. In general, latent reactive activatable groups are chemical entities that respond to specific applied external energy or stimuli to generate active species with resultant covalent bonding to an adjacent chemical structure. Latent reactive groups are those groups that retain their covalent bonds under storage conditions but that form covalent bonds with other molecules upon activation by an external energy source. In some embodiments, latent reactive groups form active species such as free radicals. These free radicals may include nitrenes, carbine or excited states of ketones upon absorption of externally applied electric, electrochemical or thermal energy. Various examples of known or commercially available latent reactive groups are reported in U.S. Pat. Nos. 4,973,493; 5,258,041; 5,563,056; 5,637,460; or 6,278,018.

For example, the commercially available multifunctional photocrosslinkers based on trichloromethyl triazine available either from Aldrich Chemicals, Produits Chimiques Auxiliaires et de Syntheses, (Longjumeau, France), Shin-Nakamara Chemical, Midori Chemicals Co., Ltd. or Panchim S. A. (France) can be used. The eight compounds include 2,4,6-tris(trichloromethyl)-1,3,5 triazine, 2-(methyl)-4,6-bis(trichloromethyl)-1,3,5-triazine, 2-(4-methoxynaphthyl)-4,6-bis(trichloromethyl)-1,3,5-triazine, 2-(4-ethoxynaphthyl)-4,6-bis(trichloromethyl)-1,3,5-triazine, 4-(4-carboxylphenyl)-2,6-bis(trichloromethyl)-1,3,5-triazine, 2-(4-methoxyphenyl)-4,6-bis(trichloromethyl)-1,3,5-triazine, 2-(1-ethen-2-2′-furyl)-4,6-bis(trichloromethyl)-1,3,5-triazine and 2-(4-methoxystyryl)-4,6-bis(trichloromethyl).

Suitable and preferred amounts of components of embolization composition components can vary. For instance, as discussed above, the relative weight of amounts of fiber material to hydrogel material suitably may vary and in certain embodiments fiber material and hydrogel material may be present in an embolization composition in relative weight amounts of 1:10 to 10:1, or more typically relative weight amounts of fiber material:hydrogel material of 2:8 to 8:2, or 3:7 to 7:3, or 4:6: to 6:4. A thermoresponsive material suitably may be will be present in an embolization composition within a relatively wide range of amounts. In certain aspects, a thermoresponsive material will not exceed about 70, 60, 55 or 50 weight percent of total solids (all components except any solvent/aqueous carrier) of an embolization composition. In certain aspects, a thermoresponsive material if utilized will be present in an embolization composition in an amount of at least about 3, 5, 10, 20, 30, 40, 45, 50 or 55 weight percent of total solids (all components except any solvent/aqueous carrier) of an embolization composition.

Preferred methods for delivery of an embolization composition disclosed herein include by injection through a catheter or syringe.

Suitable subjects for treatment with an embolization composition as disclosed herein include mammals, particularly a human. In some aspects, the subject may be identified as suffering or susceptible to a disorder or disease that would benefit from treatment with a present composition, such as undesired bleeding, or cancer. The identified subject then may be administered with an embolization composition as disclosed herein.

In certain aspects, methods are provided to treat a hemorrhage or other undesired bleeding of a subject, which may include deliver of an embolization composition as disclosed herein to the area of the undesired bleeding of the subject.

Methods for delivery of therapeutic agents to tissue of a subject are also provided and may suitably include administering to the tissue or a blood vessel associated with the tissue a sufficient amount of an embolization composition as disclosed herein to reduce blood flow from the tissue. The therapeutic agent(s) may be incorporated into the embolization composition, for example may be admixed with the hydrogel and fiber materials, or the anti-cancer agent(s) may be covalently linked to a hydrogel or fiber material.

As discussed, embolization compositions are particularly useful in treatment of cancer, including solid tumors. In such treatments, the embolization composition may be delivered at the tumor site for example via transcatheter delivery of the embolization composition.

In one aspect, embolization composition as disclosed herein is utilized for uterine fibroid embolization (UTE).

In such treatments, the administration of the embolization composition suitably produces ischemia within the tumor, resulting in tumor necrosis, for instance where 5, 10, 15, 20, 25, 30, 40, 50, 60, 70 or 80 percent or more of the mass or volume of the tumor is reduced within 1, 2, 3, 4, 5, 6, or 7 days after the first administration of the embolization composition to the subject.

In preferred aspects for such cancer treatments, the embolization composition may contain one or more anti-cancer agents, for instance the anti-cancer agent may be admixed with the hydrogel and fiber materials, or the anti-cancer agent(s) may be covalently linked to a hydrogel or fiber material. A variety of anti-cancer drugs may be incorporated into or otherwise used with a present embolization composition, including for instance, sputum agents: such as nitrogen mustard, such as nitrogen mustard, chlorambucil, cyclophosphamide (CTX), ifosfamide (IFO), etc., nitrosourea: such as N-methyl nitrosourea (MNU), ACNU, BCNU, CCNU, methyl CCNU, etc., ethyleneimine: such as 2, 4, 6-triethyleneimine triazine compound (TEM), thiotepa, formazanyl ester: Busulfan (Maliland), and dacarbazine, procarbazine, hexamethyleneamine, etc.; antimetabolites: including thymidine synthase inhibitors, such as fluorouracil (5-FU), furose fluorouracil (FT-207), Difluridine (difurfuryl FD-1), effluent (UFT), fluoroiron (5-DFUR), etc., dihydrofolate reductase inhibitors, such as methotrexate (MTX), ammonia guanidine (Bai Xuening), etc., DNA polymerase inhibitors, such as cytarabine (Ara-c), etc., nucleotide reductase inhibitors, such as hydroxyurea (HU), inosine dialdehyde (inosine dialdehyde), adenosine Dialdehyde (adenosinediialde-hgde), guanazole, etc., purine nucleotide synthesis inhibitors, such as 6-mercaptopurine (6-MP); anti-tumor antibiotics: including Cyclic antitumor antibiotics, such as doxorubicin (ADM), daunorubicin (DNR), epirubicin (EPI or E_ADM), mitoxantrone (MTT, DHAD), pirarubicin (THP) Etc., actinomycin anti-tumor antibiotics, such as actinomycin D (ACD), bleomycin anti-tumor antibiotics, such as bleomycin (seeptomycin), pingyangmycin (A5), etc. Mitomycin anti-tumor antibiotics, such as mitomycin A, mitomycin B, mitomycin C (MMC), etc., phlomycin such as phosfomycin (MTH), olive mold, and streptozotocin (STT); anti-tumor botanicals: vinblastine and taxanes that inhibit microtubule and tubulin polymerization, such as vinblastine (VLB), vincristine (VCR), Vinblastine amide (VDS:), norvinine (NVB), paclitaxel (PTX), taxotere, etc., topoisomerase inhibitors such as camptothecin and podophyllotoxin, such as camptothecin (CPT)), hydroxycamptothecin (HCPT), etoposide (Etoposide, VP-16), agents that inhibit DNA synthesis of tumor cells, such as harringtonine and indirubin; and other agents such as cisplatin (DDP), carboplatin (CBP), platinum oxalate (oxaliplatin, L-OHP). Preferred anti-cancer agents to use with an embolization composition may include paclitaxel, 5-FU, epirubicin, cisplatin, vincristine, docetaxel and etoposide.

Such anticancer agents may be incorporated into an embolization composition in a recommended dosage amount for the particularly agent.

As discussed above, a present composition also may comprise one or more thrombogenic agents and/or thrombosis agents to thereby deliver such therapeutics to a patient. Such thrombogenic agents and thrombosis agents thrombosis agents may be incorporated into an embolization composition in a recommended dosage amount for the particular agent. Exemplary thrombogenic agents may include heparin, warfarin, dabigitran, apixaban, rivoraxaban and edoxaban.

Other therapeutic agents also may be included into a present composition.

In related aspects, methods are provided comprising administration of an embolization composition as disclosed herein for treating skin, head, or neck tumors, tumors of the uterus or fallopian tubes, liver or kidney tumors, endometriosis, or fibroids. Methods are also provided comprising administration of an embolization composition as disclosed herein for treatment of renal angiomyolipomas and renal cell carcinoma; for treatment of cerebral and intracranial aneurysms, neuroendocrine metastases, intracranial dural arteriovenous fistula and patent ductus arteriosus. Methods are further provided comprising administration of an embolization composition as disclosed herein for hepatic artery embolization and pulmonary artery embolization. Methods are further provided comprising administration of an embolization composition that comprises one or more thrombosis agents to thereby treat a subject.

The following non-limiting examples are illustrative of the invention.

### General

The following materials and methods were used in the examples which follows:

### Materials and Methods

### Materials

Sodium alginate with high guluronic acid content and medium viscosity (I-1G) was purchased from Kimica Corporation. 2-Aminoethyl methacrylate hydrochloride (AEMA) was purchased from Polysciences. N-(3-Dimethylaminopropyl)-N′-ethylcarbodiimide hydrochloride (EDC), N-hydroxysuccinimide (NHS), 2-Morpholinoethanesulfonic acid monohydrate (MES acid), 4-Morpholineethanesulfonic acid sodium salt (MES sodium salt), ammonium persulfate (APS), N,N,N′,N′-Tetramethylethylenediamine (TEMED), N-Isopropylacrylamide (NIPAM), acrylic acid, caffeine, deuterium oxide (D₂O), methanol-d4, activated charcoal, and poly(ε-caprolactone) were purchased from Sigma-Aldrich.

### Alginate Purification

I-1G alginate was purified through dialysis and charcoal filtering. See FIG. 3. The alginate was dissolved at 1% w/v in diH₂O and dialyzed in 3.5 kDa MWCO dialysis membrane for 2 days. The dialysis water was changed 3 times a day. Following dialysis, 0.5 g of activated charcoal per gram of alginate was added to the solution and stirred for 30 minutes. The solution was then allowed to settle for 0.5-1 hr. Charcoal was filtered out using a 0.22 μm PES vacuum filter. The solution was then frozen overnight and lyophilized for 4 days.

### Alginate Methacrylation

Purified alginate was dissolved in 0.1 M MES buffer (pH 6.4). NHS and EDC were added to the solution and left to stir for 5 minutes before AEMA was added. The solution was shielded from light and allowed to react for 17 hours in at 2° C. For 30% methacrylation, the AEMA:EDC:NHS:Alginate mass ratio was 2.24:2.8:1.3:1, while for 20% methacrylation, the ratio was 1.12:1.4:0.75:1. The solution was then dialyzed against 137 mM at 2° C. for one day to remove unreacted AEMA as well as urea byproducts of the carbodiimide reaction (FIG. S3). The dialysis solution was changed one. The solution was then dialyzed in 3.5 kDa MWCO dialysis membrane against water for 2 days at 2° C., with the dialysis water changed 3 times a day, before being frozen overnight and lyophilized for 4 days. Methacrylation degree was quantified using NMR using caffeine as an external standard using D₂O as a solvent. The methacrylated alginate was shielded from light and stored at 2° C. and used within 2 weeks.

### PCL Electrospinning

A 16% (w/w) PCL solution was prepared using PCL with a molecular weight of 80,000 and 45,000 at a mass ratio of 85:15 in a solvent of DCM and DMF with a mass ratio of 9:1 and was placed on a shaker at a rate of 200 rpm at room temperature overnight. The solution was placed in a 6 mL syringe with a metallic needle of 27G with a flat tip. The solution was electrospun onto a circular plate with a 30 cm diameter spinning at 900 rpm at a feeding rate of 2.5 mL/h at a distance of 12 cm and a voltage of 16 kV. A cover slip was used to collect some fibers, which were observed using a light microscope to confirm that no fiber beading was present. After electrospinning, fiber sheets were collected onto 9 mm adhesive rings.

### PCL Plasma Treatment

PCL sheets were plasma treated using an Expanded Plasma Cleaner (PDC-001, Harrick Plasma) to introduce COOH groups onto the fiber surface. The plasma chamber was first cleaned by purging with oxygen twice, reducing pressure to 300 Torr, and turning the RF power on to high for 10 minutes. Fiber sheets were then loaded two at a time into the chamber and secured in place by taping down the adhesive rings. The cleaning procedure was repeated with the fibers in the and repeated after the sheets were flipped.

### TBO Assay

An 8 mm diameter punch was used to collect samples of the fiber sheets to quantify plasma treatment efficiency. The punched fiber sheets were placed in a well and washed with 1 mL of 0.1 mM NaOH twice. 1 mL of 0.5 mM TBO in 0.1 mM NaOH was added to each well. The sheets were left on a shaker at 200 rpm and left at room temperature of 12 h. The TBO solution was removed and the sheets were washed with 1 mL 0.1 mM NaOH. 1 mL of 50% (v/v) acetic acid was added to each well. The sheets were then left on a shaker at 200 rpm for 30 min. 100 μL of the supernatant was transferred to a 96-well plate. Wells of 5 μM, 50 μM, and 250 μM TBO in 50% (v/v) acetic acid were prepared in triplicate for a standard curve. The plate was measured at 633 nm using a microplate reader and the COOH concentration was calculated based on the standard curve.

PCL Functionalization 0.1M MES buffer (pH 6.4) was prepared. Using a molar ratio of 25:25:10:1 of NHS:EDC:AEMA:COOH, NHS and EDC were added to the MES solution, and 10 mL were transferred to a Petri dish containing a PCL fiber sheet. AEMA was dissolved in 5 mL MES buffer and added to the Petri dish after 30 min. The Petri dishes were left on a shaker at 200 rpm at room temperature for 12 h. The reaction solution was removed, and the sheets were washed with 15 mL 70% (v/v) ethanol then 15 mL diH₂O three times, protected from light, and left in a hood to dry. The efficiency of the reaction was determined by another TBO assay.

### Fiber Fragmentation

The MA-PCL sheets were washed with 10 mL isopropyl alcohol (IPA). The sheets were dipped in liquid nitrogen in a mortar and fragmented first using scissors, then with a pestle. 10 sheets were added to a large cryovial and 10 mL 50% (v/v) ethanol was added. The cryovial was placed in liquid nitrogen to freeze the fiber dispersion before a grinding bar was added. The fibers were then pre-cooled for 3 minutes before 6 cycles of grinding for 1 minute with 2 minutes of cooldown in between using a grinding rate of 6 in a 6770 Freezer/Mill (SPEX SamplePrep). Following milling IPA was added to the cryovial to collect the fiber fragments in a 50 mL centrifuge tube. The fiber dispersion was filtered using a 100 μm nylon filter and the filtrate was centrifuged for 5 min at 3000 rpm and the IPA was drained. The tube was left open, protected from light, in a hood to dry overnight. MA-PCL was protected from light and stored at −20° C.

### NIPAM Nanogel Synthesis

48 mL of 65 mM solution of NIPAM, AAc, and MBA was prepared with a molar ratio of 40:9:1 NIPAM:AAc:MBA. The solution was transferred to a clean round bottom flask and purged with nitrogen for 25 min. 1 mL 10 mg/mL SDS and 1 mL 10 mg/mL APS solutions were prepared and added to the reaction vessel. The solution was purged with nitrogen for an additional 5 minutes. The reaction vessel was then placed in a 65° C. oil bath to react for 3 hours. The solution was dialyzed against diH₂O for 1.5 days with the dialysis water changed 3 times. 5 mL of solution was lyophilized for two days to determine a concentration. The solution was then concentrated to roughly 30 mg/mL NNG using a rotary evaporator. Assuming all the AAc added was incorporated into the NNGs, a solution with a ratio of 1:1:1 AAc:AEMA:EDC was prepared and allowed to react for 7 hours. The solution was then dialyzed in a 12-14 kDa dialysis membrane against 137 mM NaCl for 12 hr then against diH₂O for 24 hours, with the dialysis water changed 3 times. Based on final volume following rotary evaporation ˜15 mg MA-NNGs were lyophilized. NMR analysis performed using methanol-d4 as the solvent.

### Cryogelation

Alginate was dissolved in degassed diH₂O at 1.11× the desired weight percent, to reserve 10% of the final volume for initiator solutions. If fiber composites were being prepared, the fibers were first dispersed in water and alginate was dissolved in the dispersion. If NNGs were being used, the alginate was dissolved directly in the NNG dispersion. The solution was cooled to 2° C. APS and TEMED were each prepared in cooled water with 5% of the final volume. The solutions were prepared such that, when added to the alginate solution, the concentration would be 0.2% w/v. APS was added to the pre-cooled alginate and the solution was thoroughly mixed. TEMED was then added to the solution, thoroughly mixed, and quickly added to Teflon molds that had been pre-cooled to −20° C. After aliquoting, the molds were quickly transferred to a −20° C. freezer and left to react for 24 hours. After 24 hours, the gels were removed from the molds and washed with water.

### Example 1

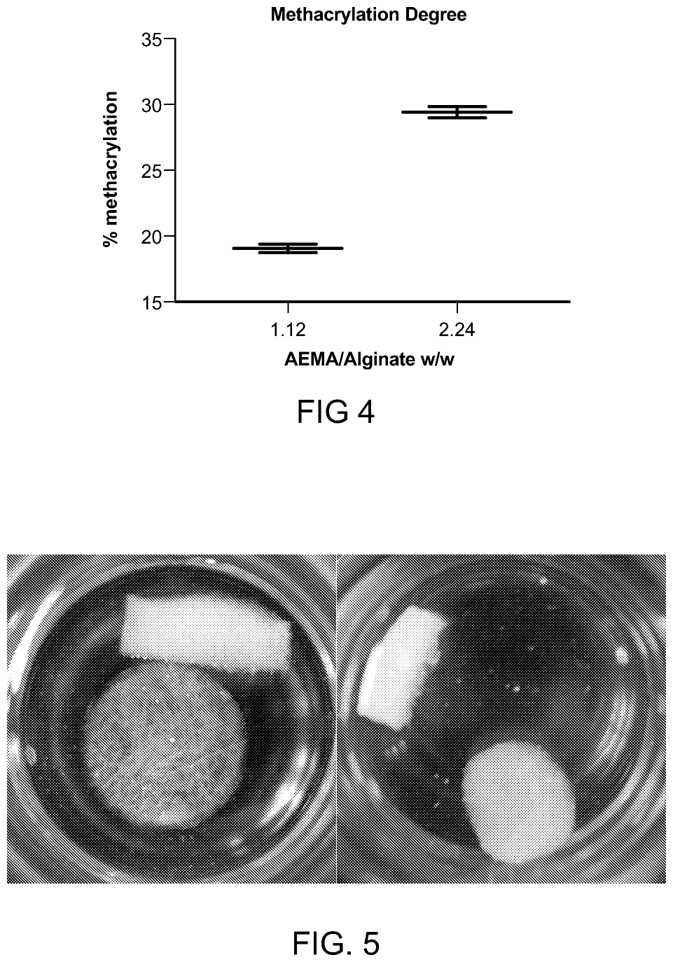

Alginate was functionalized with pendant methacryloyl groups in order to introduce polymerizable groups for cryogelation and interfacial bonding. See FIG. 3. The methacrylation reaction was confirmed by FTIR and quantified by ¹H NMR using caffeine as an external standard. The degree of methacrylation could be consistently controlled by adjusting the mass ratio of alginate to 2-aminoethyl methacrylate (AEMA) during the carbodiimide functionalization reaction. See FIG. 4. At the two alginate:AEMA ratios tested, which were tested in triplicate, roughly 20% and 30% of the alginate repeat units were functionalized with methacryloyl groups. In order to quantify the methacrylation degree, it was assumed that the alginate was completely pure, with each repeat unit having an empirical formula of NaC₆H₇O₆.

Macroporous alginate samples were synthesized by radical polymerization of MA-alginate solutions of varying w/v ratios at −20° C., hereafter referred to as alginate loading percent. The resulting macroporous structure was easily visible compared to alginate gels formed at room temperature. See FIG. 5.

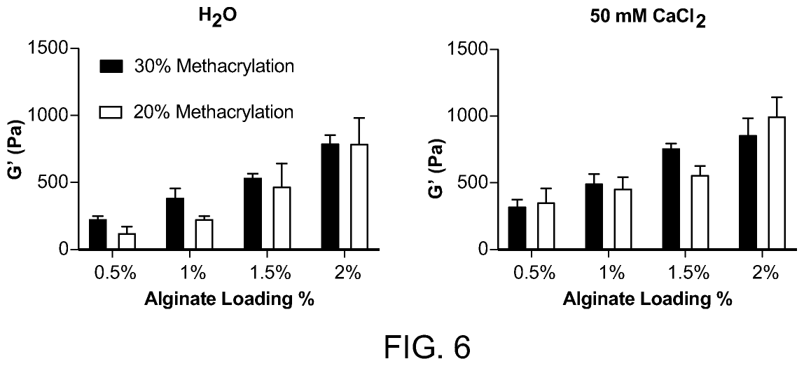

In order to quantify the effect of methacrylation degree on the resulting cryogel properties, alginate cryogels of the two methacrylation degrees prepared at various loading percentages and their rheological properties were measured. See FIG. 6. The 30% methacrylated alginate cryogels were 11.4% stiffer than their 20% methacrylated counterparts in water, but only 2.84% stiffer on average in a 50 mM CaCl₂ bath. This was expected, as the relative importance of the degree of covalent crosslinking is lessened in a dual-crosslinked structure. The 20% methacrylated alginate was not explored further.

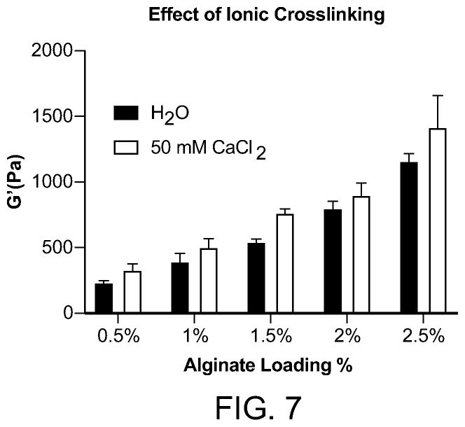

The effect of ionic bonding on cryogel mechanical properties was further quantified by rheology at various alginate loading percentages and was shown to result in an average increase in storage modulus (G′) of 25.2%. See FIG. 7.

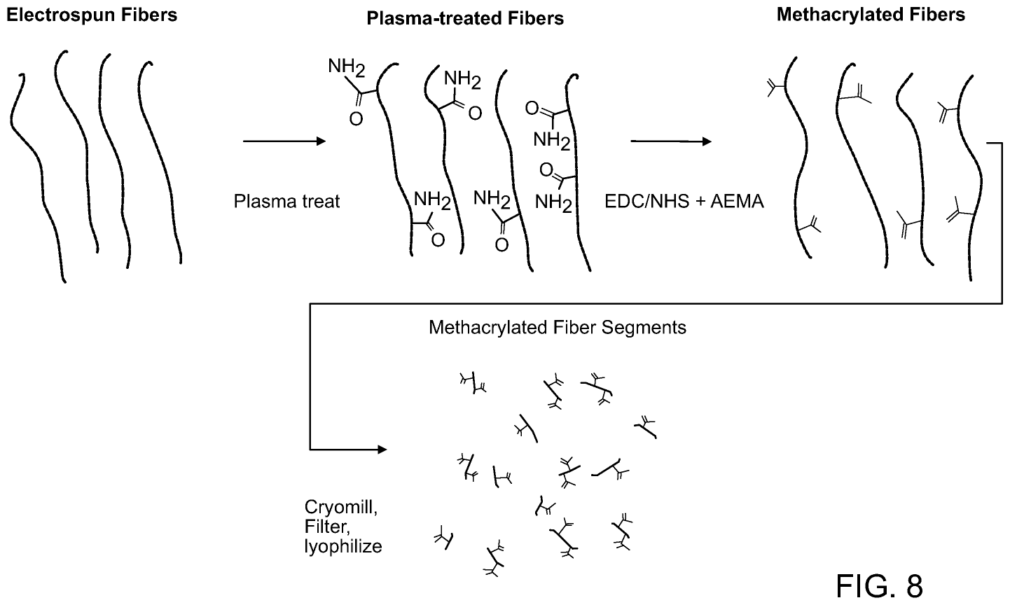

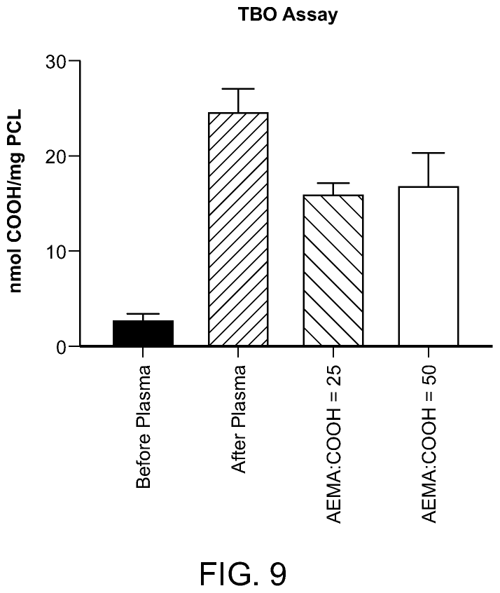

Electrospun PCL fibers were plasma-treated to introduce carboxylic acid (COOH) groups, which were subsequently functionalized with AEMA by carbodiimide chemistry. See FIG. 8. A toluidine blue 0 (TBO) assay was used to measure the degree of COOH functionalization following plasma treatment, as well as the decrease in COOH concentration. In order to quantify the degree of methacrylation, all lost COOH groups following methacrylation were assumed to have reacted with AEMA. The difference in the degree of functionalization of the fibers was shown to be statistically insignificant at the tested AEMA:COOH ratios, with a total average degree of functionalization of 8.2 nmol AEMA per mg PCL. See FIG. 9. The functionalized fibers were fragmented in a cryomill before being incorporated in alginate as a composite material.

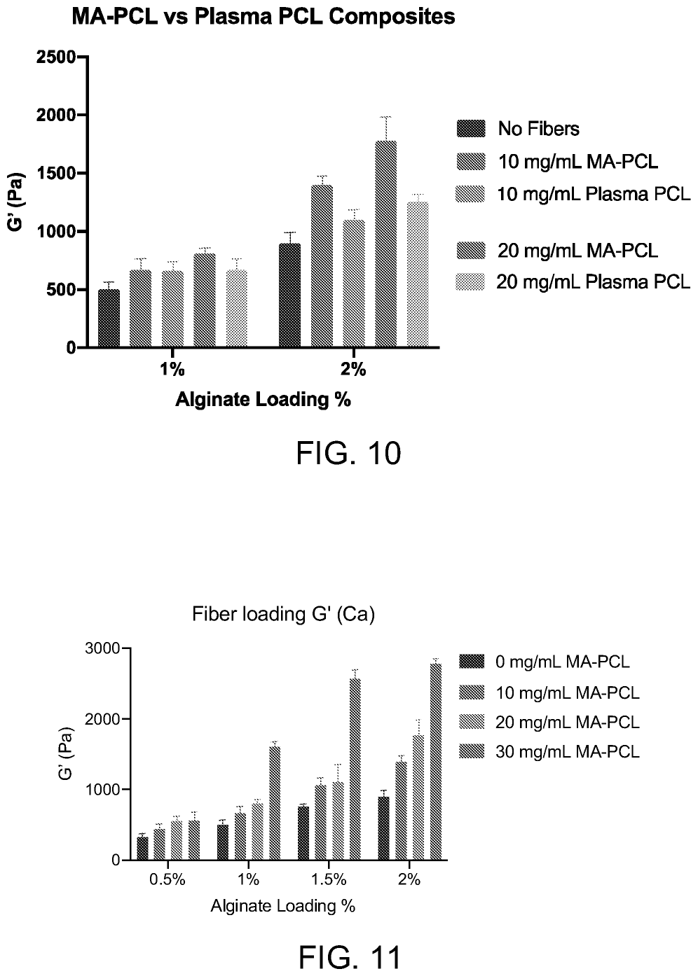

Fragmented AEMA-functionalized fibers (MA-PCL) and plasma-treated PCL (Plasma PCL) were dispersed in alginate cryogels and crosslinked in a 50 mM CaCl₂ bath to quantify the effect of interfacial bonding on mechanical properties by rheology. See FIG. 10. The average increase in G′ for the tested fiber loading ratios was higher for MA-PCL composites than for plasma PCL composites, with an average increase in G′ of 66.0% and 31.6% respectively.

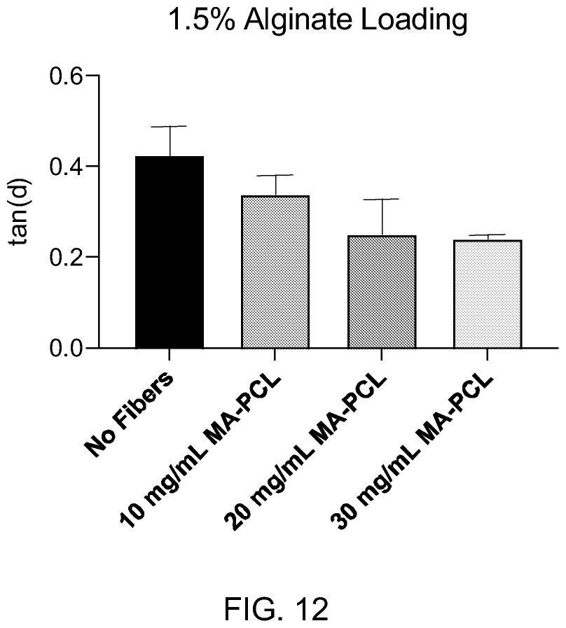

The MA-PCL composites were further explored using rheology to determine the effect on mechanical properties at a greater number of alginate loading percentages and fiber loading percentages in 50 mM CaCl₂. See FIG. 11. For all tested samples, G′ increased with the addition of MA-PCL. Interestingly, there was a large jump in G′ 1%, 1.5% and 2% alginate loading when MA-PCL was loaded at 30 mg/mL. In addition, an increase in MA-PCL loading was associated with a decrease in tan(δ), showing that the elasticity of the composite also increased. See FIG. 12.

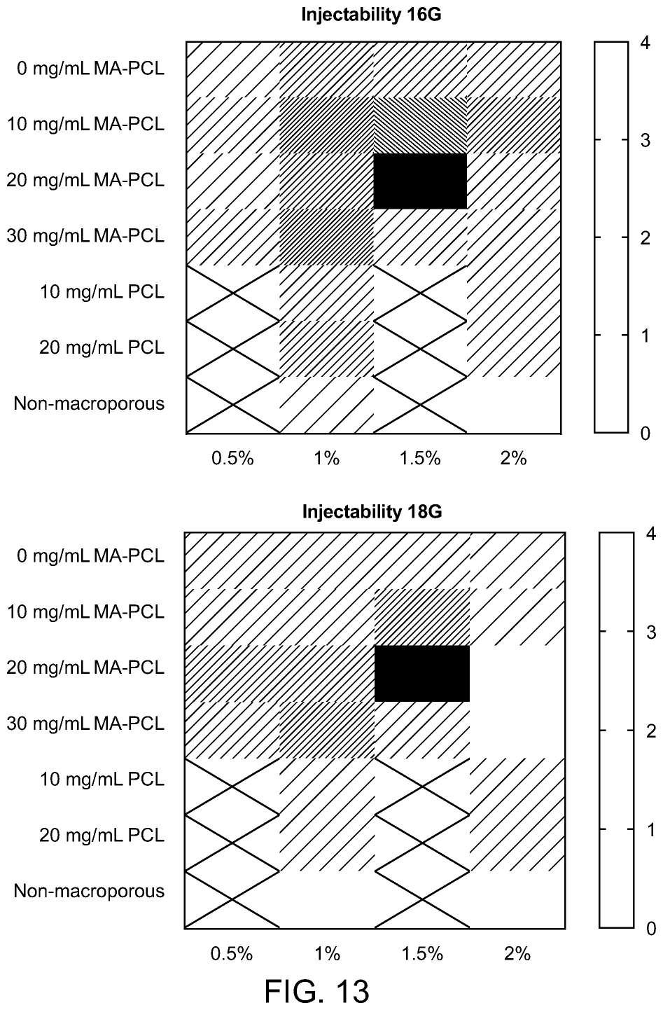

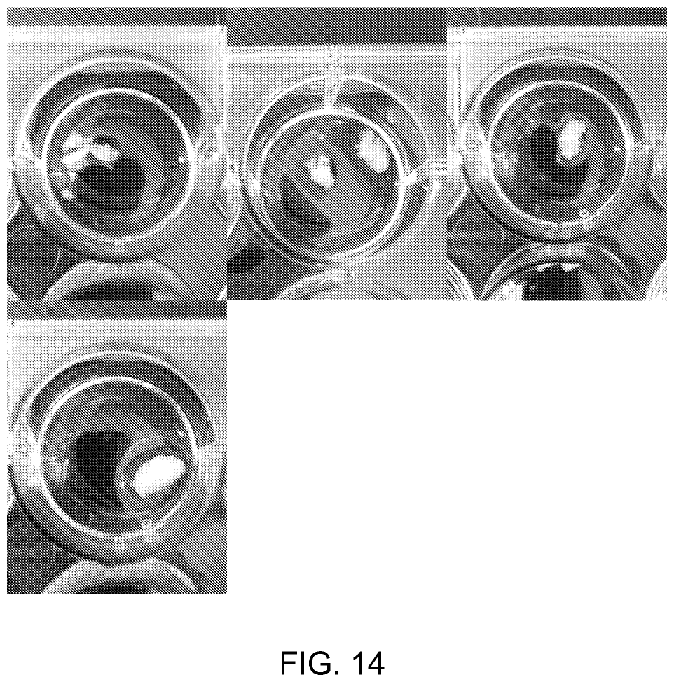

As a proxy for catheter compliance, the cryogel composites were ionically crosslinked in a 50 mM calcium bath and injected through 16G and 18G needles in triplicate and qualitatively judged on a scale of 0 to 4. See FIG. 13. A 100 mM bath was also used, but no gels with appropriate mechanical properties were able to be injected (data not shown). The scores were given based on the criteria in Table 1 below and representative images of each of the scores can be found in FIG. 14. In both the 16G and 18G needle tests, the 1.5% alginate loading with 20 mg/mL MA-PCL samples passed through the needle with the least amount of visible fracture.

TABLE 1
Injectability scoring criteria

| Score | Result |
|---|---|
| 0 | Unable to pass through the catheter |
| 1 | Broken into multiple pieces following injection |
| 2 | Broken into two pieces following injection |
| 3 | Visibly damaged, but did not completely fracture |
| 4 | No visible damage following injection |

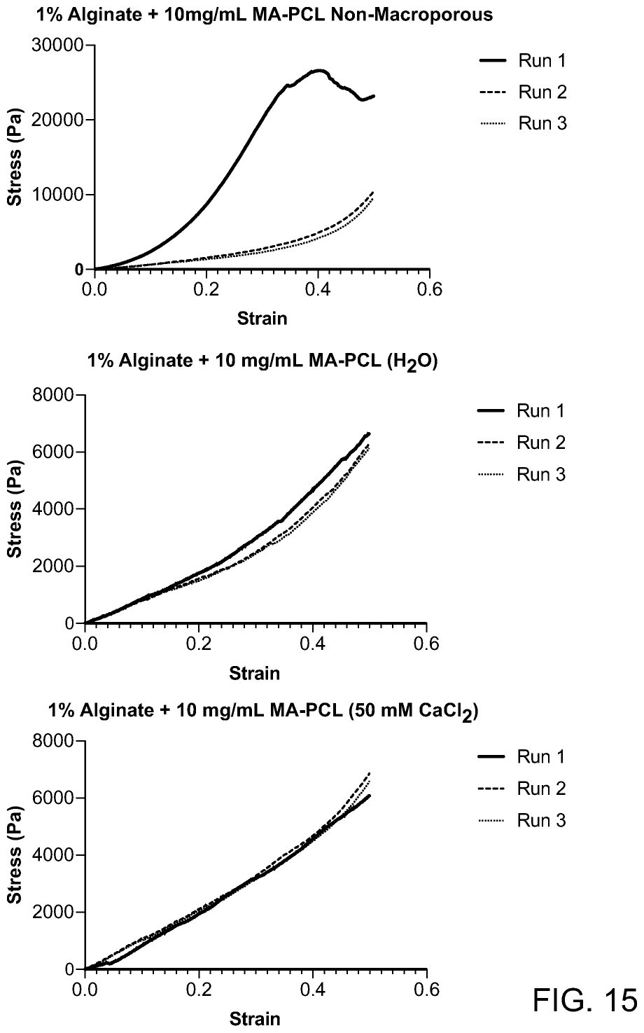

In order to compare the toughness of macroporous and non-macroporous gels, as well as covalently crosslinked and dual-crosslinked composites, select formulations were compressed to 50% strain 3 times, and each of the stress-strain curves were measured. See FIG. 15. It can be seen from the stress-strain curves that the macroporous gel cannot deform elastically to 50%, displaying a clear fracture point at 27.2% strain. A slight decrease in Young's modulus following iterative compression to 50% in the covalently crosslinked macroporous composite was observed, while the addition of ionic crosslinks resulted in an increase in toughness.

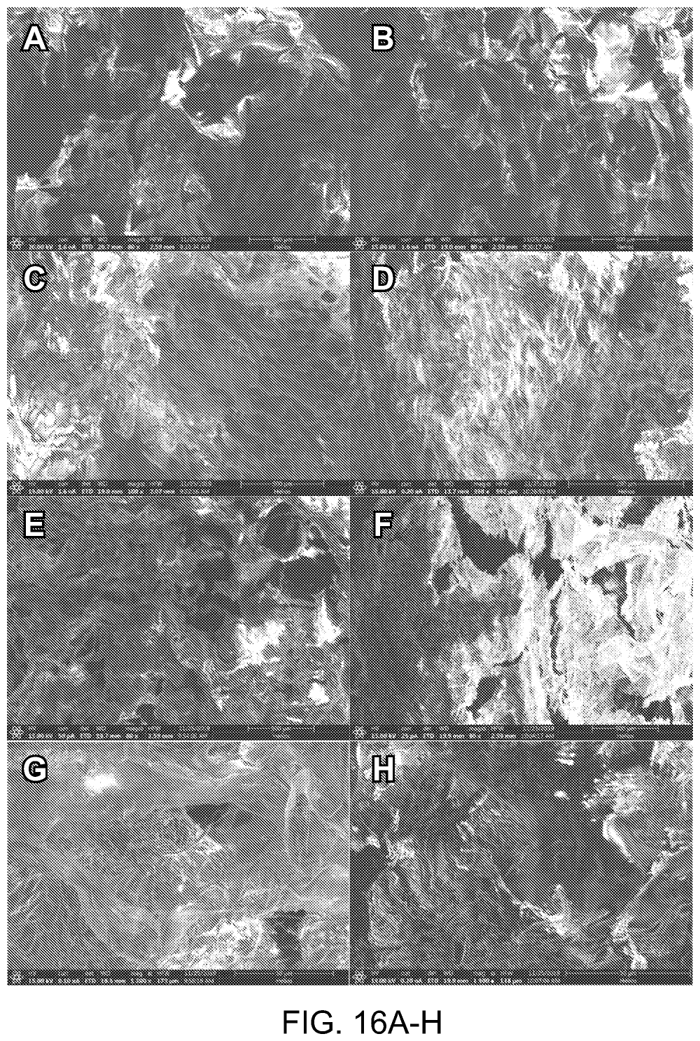

Various samples were imaged using SEM. See FIG. 16. The samples were prepared by drying using ethanol and hexamethyldisilazane (HDMS), rather than vacuum drying or lyophilization, so as not to disrupt the pore structure. A general trend of decreasing pore size with increasing alginate loading percent can be observed. Furthermore, the pore size and morphology of the 2% alginate cryogel is significantly different than that of the lower loading percentages.

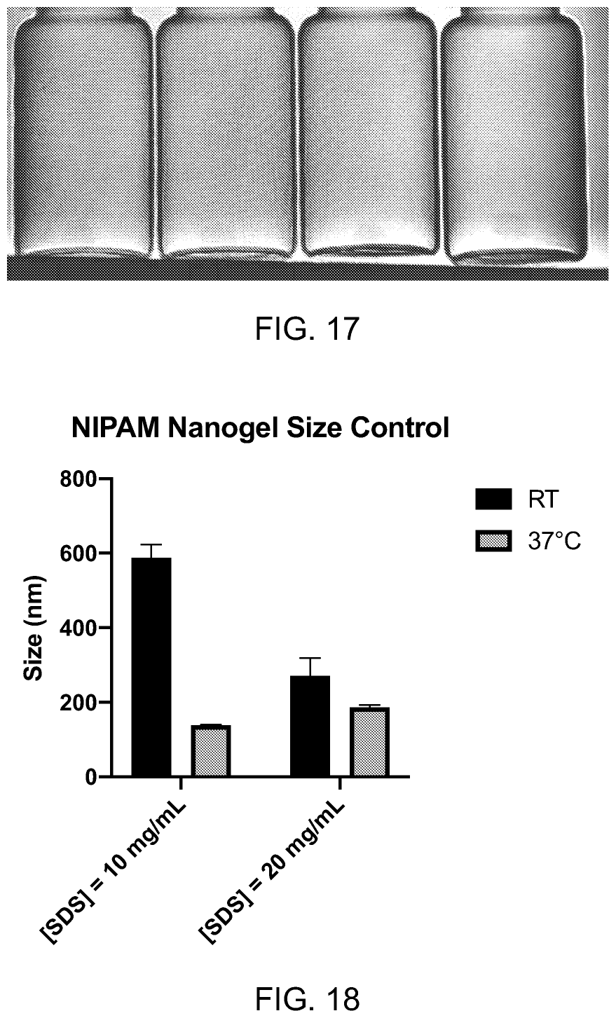

The radiopacity of the cryogel composites with various barium crosslinking degrees was investigated. See FIG. 17. 1% alginate cryogels with 10 mg/mL fibers were placed in 50 mM ionic solutions of varying Ca²⁺ and Ba²⁺ ratios. As expected, the radiopacity appears to increase with increasing barium concentration, but the differences are subtle.

### Example 2: Engineering a Thermoresponsive Macroporous Composite

A thermoresponsive analogue of the macroporous alginate cryogel was designed to enhance its efficacy as an embolization device by an in situ stiffening response. NIPAM and

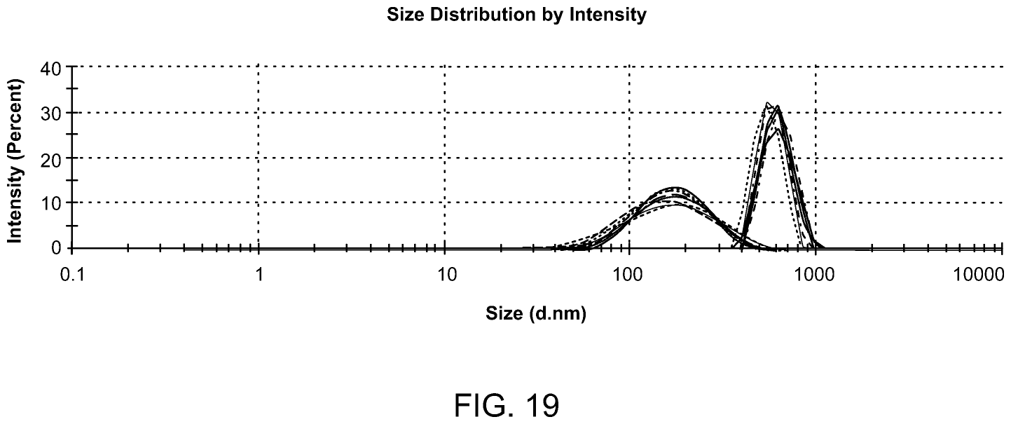

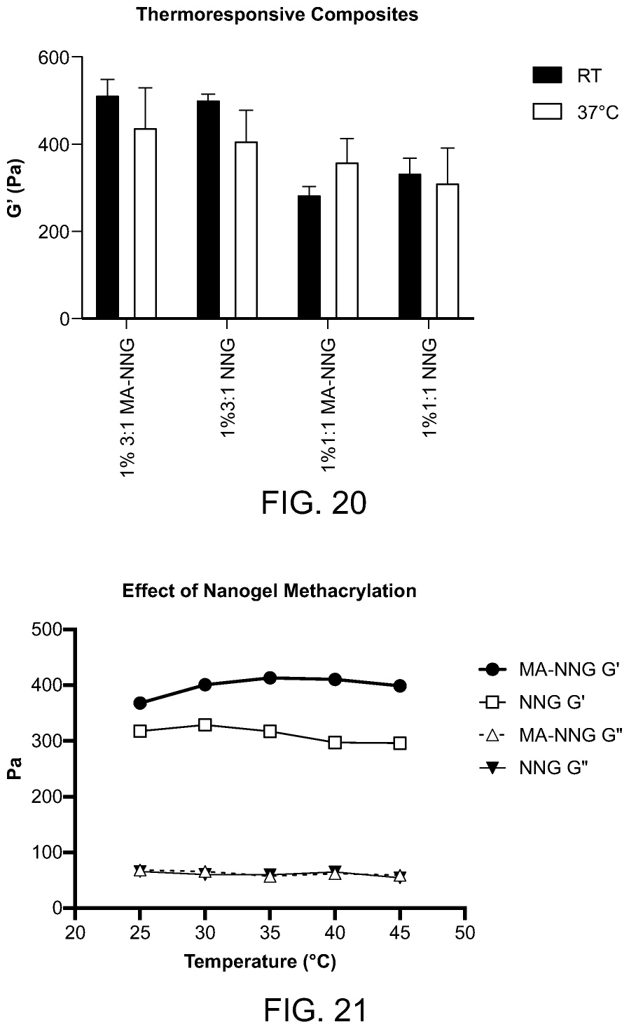

AAc were co-polymerized in the presence of MBA and sodium dodecyl sulfate (SDS) to form Poly(N-isopropylacrylamide) nanogels (NNGs). These nanogels were then functionalized with AEMA by carbodiimide chemistry (MA-NNG), before being incorporated into the alginate cryogels. Methacrylation was confirmed by NMR, which showed characteristic vinyl peaks at 6.1 ppm and 5.6 ppm. See FIG. 18. The gels were characterized at room temperature and at 37° C. by dynamic light scattering (DLS), which revealed that the average nanogel size decreased from roughly 500 nm to 100 nm upon heating. See FIG. 19. In this set of experiments, the overall polymer loading percentage was set to 1% or 1.5% with alginate-to-NIPAM mass ratios of 3:1 or 1:1. Both MA-NNG composites and non-functionalized NNG composites were investigated. The thermoresponsive properties of these Alginate-NIPAM composite cryogels were tested by temperature sweep rheology in triplicate. Of the four formulations tested, only the 1% polymer loading with 1:1 alginate-to-MA-NNG samples exhibited a stiffening response at elevated temperatures. See FIG. 20. Representative temperature sweep curves clearly show that at the same polymer loading percentage and alginate-to-NIPAM mass ratio, the MA-NNG composite showed a stiffening response between 30° C. and 35° C., with a G′ increase of roughly 12% while non-functionalized NNG composites became softer by around 3%. See FIG. 21. Furthermore, the loss modulus (G″) remained relatively constant over this temperature range, resulting in a decrease in tan(6), indicating an increase in elasticity. Rheological temperature sweeps measurements of 1.5% polymer loading with the same 1:1 alginate-to-NIPAM ratios were also performed, but did not exhibit the same stiffening effect at elevated temperatures.

### Example 3: Fiber Sintering as a Stiffening Mechanism

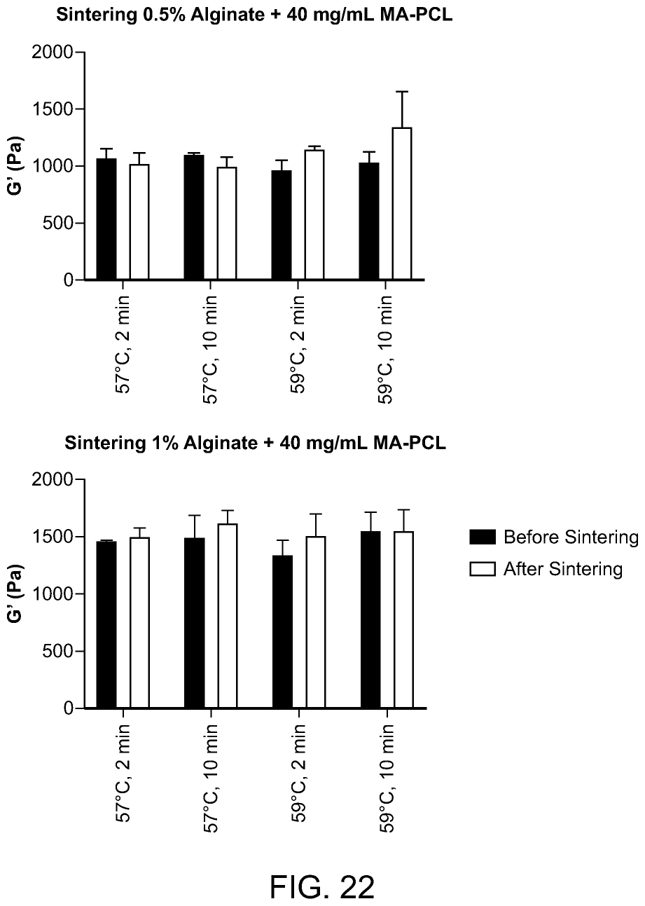

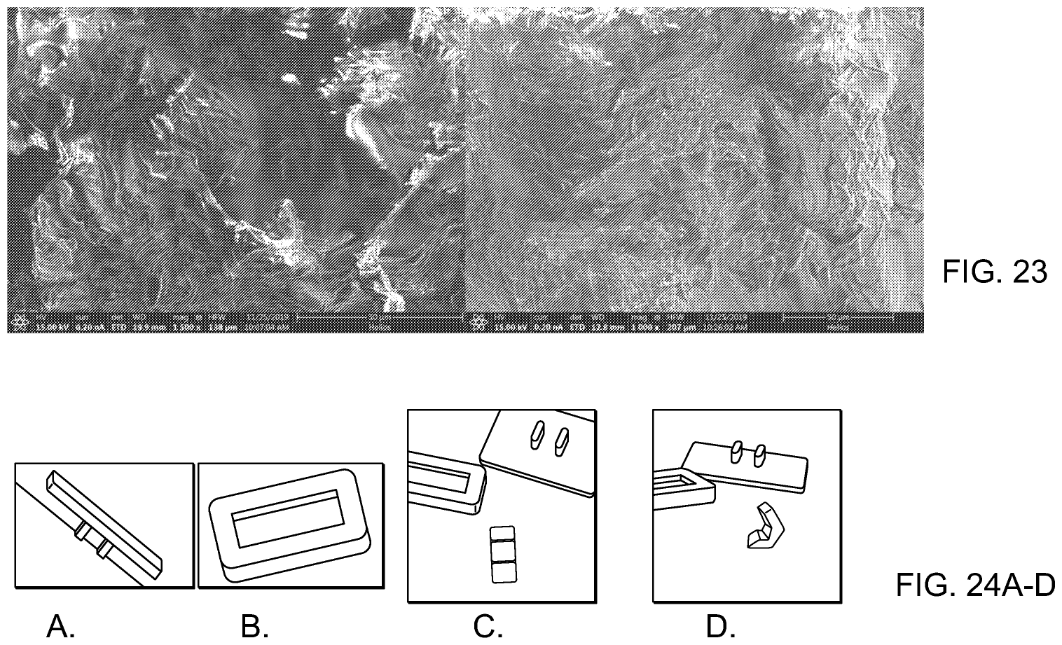

Cryogel composites with low alginate loading percentage and high MA-PCL loading were prepared in order to maximize the chance of fiber-fiber contact. The gels were characterized by rheology before and after sintering at different times and temperatures. See FIG. 22. However, none of the composites demonstrated statistically significant strengthening under any of the tested sintering conditions. SEM micrographs of 1% alginate and 30 mg/mL MA-PCL were also taken before and after exposure to a 57° C. water bath for 10 minutes and showed no discernable differences. See FIG. 23.

### Discussion

Macroporous Alginate-PCL Composites with Interfacial Bonding as an Embolic Agent

A series of novel macroporous composites have been synthesized and tested for suitability for use as an embolization agent. Alginate was selected as the hydrogel matrix material due to the fact that it is biocompatible, affordable, and is crosslinked by divalent cations. Alginate was purified by charcoal filtering and dialysis before being methacrylated. Each repeating guluronic and mannuronic acid group of alginate possesses a carboxylic acid, which can be converted to a methacrylate group by a carbodiimide reaction with the amine group of AEMA. The pendant methacrylate groups can then be radically polymerized to covalently crosslink alginate. If the alginate precursor solution is cooled to near freezing before initiation, and only a small amount of initiator is used, the solution can be frozen prior to significant covalent crosslinking. As the solution freezes, ice crystals nucleate and grow, resulting in regions of concentrated alginate between the ice crystals. These regions polymerize and when the ice crystals thaw, the macroporous structure remains. See FIG. 1. These macropores are able to collapse, which allows cryogel structures to be elastically compressed far more than hydrogels of the same polymer loading percentage which are not crosslinked in the presence of a porogen. This phenomenon was observed in the iterative compression tests, where macroporous and non-macroporous alginate composites were compressed to 50% strain. See FIG. 15. The stress-strain curve of the initial compression run of the non-macroporous composite shows fracture at 27.2%, followed by a significantly decreased compressive modulus in the successive runs.

By submerging the alginate cryogels in a bath of divalent cations, a dual-crosslinked structure can be achieved. This not only increases stiffness (FIG. 7), but also results in a tougher structure. This increase in toughness can be attributed to the fact that ionic bonding is a caused by electrostatic interactions between divalent cations and negatively charged carboxylic acid groups on the alginate backbone, meaning they can break and reform. Conversely, covalent crosslinks cannot reform once broken. When subject to significant loads, such as those experienced during transcatheter delivery, these electrostatic interactions act as sacrificial bonds, breaking to relieve internal stresses. Upon removal of the load, the ionic crosslinks can reform, resulting in minimal loss of mechanical properties. The enhanced toughness of dual-crosslinked structures was also observed in the iterative compression test, where signs of fracture could be observed in the initial run of the covalently crosslinked cryogel, resulting in a slight decrease in the compressive modulus in the following runs, whereas the dual crosslinked cryogels had no discernable decrease in mechanical properties following compression to 50% strain.

In order to overcome the competing requirements of stiffness and injectability, composite stiffening was employed. Comparing cryogels with 2% alginate loading to macroporous composites with 1% alginate loading and 20 mg/mL MA-PCL, which have similar average G′ values of 897.6 Pa and 807.2 Pa respectively, the macroporous composite had an average injectability score of 2.5 from both the 16G and 18G, while the 2% alginate loading cryogel had an average injectability score of 1.3. This significant difference in injectability can be attributed to the fact that the incorporation of nanofibers did not significantly affect the pore morphology. Comparing the SEM micrographs, the composite had large pores, visually similar to those of the 1.5% loaded cryogel, while the 2% loaded cryogel had pores that were much smaller (FIGS. 16 D, F). The compressibility of macroporous gels results from pores being able to collapse, rather than the actual deswelling of the polymer matrix, allowing composites of to have improved compressibility compared to cryogels of similar mechanical properties, based on differences in pore size and morphology.

The large jump in storage modulus at 30 mg/mL MA-PCL loading was not expected (FIG. 11). The SEM micrographs reveal that the pore morphology of the 1% alginate loading composites differ significantly for 20 mg/mL and 30 mg/mL MA-PCL (FIGS. 16 E,F). Specifically, the pores appear smaller and more angular in the composite with more MA-PCL. Upon closer inspection, the fibers appear to form a near-contiguous surface at the higher loading percentage, coating the hydrogel matrix (FIGS. G,H). The higher fiber concentration on the surface may indicate that the hydrogel matrix has been saturated with fibers, causing them to accumulate more on the outside of the gel. It is possible that this stiff coating of fibers is responsible for the jump in mechanical properties.

Curiously, this dramatic jump in storage modulus was not observed for the 5% alginate loading composite. Assuming methacrylated alginate is completely pure, in other words, that methacrylated guluronate and mannuronate groups have an empirical formula of NaC₁₂H₁₆O₇N non-functionalized groups have an empirical formula of NaC₆H₇O₆, a 30% methacrylation degree implies a 1296 nmol/mg methacrylate concentration. Even accounting for the higher density of PCL, the methacrylate concentration of alginate is significantly higher than that of PCL at 2.1 mmol/cm³ compared to 9.2 μmol/cm³. Its dramatically higher degree of functionalization and softer mechanical properties means alginate can be thought of as the glue holding the composite together. In other words, an increase in fiber-fiber contact at the expense of fiber-alginate contact would likely decrease mechanical properties. Therefore, it is possible that at significantly low alginate-to-PCL ratios, fibers, even when methacrylated, can disrupt the structural cohesiveness of the composite and detract from mechanical properties. If this is the case, the threshold ratio is between 0.5% alginate:30 mg/mL MA-PCL and 1% alginate:30 mg/mL MA-PCL.

Based on the injectability tests, the most promising formulation for use as an embolization device is the 1.5% alginate loading with 20 mg/mL MA-PCL. Currently available polymer-based embolization devices have Young's moduli ranging from 10 kPa to 50 kPa. It was found from the compression tests that the 1% alginate loading with 10 mg/mL MA-PCL had a Young's modulus (E) of 10 kPa, meeting the stiffness requirements for embolization. Based on rheological measurements, our candidate formulation was 67% stiffer than the E=10 kPa composite, putting its mechanical properties firmly in the appropriate range.

Furthermore, existing polymer-based embolization devices are rated to withstand compressive strains of up to just 33% during delivery, severely limiting the maximum size of gel that can pass through microcatheters. The samples used to inject through an 18G needle were quarter cylinders with a radius of 4 mm and a height of 3 mm, making their largest dimension 6.4 mm and volume 37.5 μL. The largest available size of Embospheres have particle diameters ranging from 900-1200 μm, corresponding to an average volume of 0.6 μL. The 18G needles used to test injectability and catheter compliance have an inner diameter of 0.033″ or 0.838 μm, which is smaller than the both the microcatheters used for the largest Embospheres, which have inner diameters of 0.035″ or 0.038″. Ignoring the morphological differences, this data suggests that the volume of our macroporous composite that is able to be delivered through these microcatheters is over 70 times greater than the largest Embospheres, allowing our formulation to be used to target a much wider range of blood vessel sizes.

In addition, the stress-strain curve of a representative macroporous composite formulation (FIG. 15, bottom right) shows that the linear elastic region extends to 45% strain, which suggests that the composites can be used for a blood vessels with a much wider range of diameters compared to platinum coils, which must be carefully selected to have a coil diameter 20% larger than blood vessel. Moreover, the proposed composite embolization material would occlude the blood vessel by re-expansion, rather than re-coiling to a pre-determined shape, making it suitable for a wide range of vessel morphologies.

In summary, the macroporous composites investigated possess sufficient mechanical properties for embolization, can withstand injection through needles smaller than relevant microcatheters, are suitable for a wide range of blood vessel sizes and morphologies, present no risk of catheter entrapment, are radiopaque, and—being alginate-based—can be very affordable.

### Thermoresponsive Macroporous Composites for In Situ Stiffening

Due to the fact that existing embolization devices are often delivered in cooled saline, a thermoresponsive analogue of the macroporous composites was investigated to improve their efficacy as embolic materials by taking advantage of the temperature difference during delivery and in situ, allowing gels to be soft during delivery, allowing for catheter compliance, and stiffen in the blood vessel to improve stability. NIPAM is a commonly used thermoresponsive biomaterial and exhibits LCST behavior at ˜32° C. In other words, as temperature rises above 32° C., NIPAM becomes hydrophobic and crashes out of solution. When polymerized into nanogels, heating above LCST causes hydrophobic interactions between strands of PNIPAM, resulting in the collapse of the particle (FIG. 19).

At 1% polymer loading with an alginate-to-NIPAM mass ratio of 1:1, MA-NNG composites induced a stiffening effect, causing G′ to increase 12% at physiological temperatures compared to at room temperature. Conversely, non-functionalized NNGs softened slightly across the same temperature range, while G″ of both composites remained relatively constant. MA-NNG composites are covalently incorporated into the alginate matrix during cryogel polymerization, allowing them to pull alginate strands closer together upon nanogel collapse, stiffening the walls of the macroporous structure and making them more resistant to plastic deformation, indicated by the decrease in tan(6) (FIG. 22). Without the functionalization and the corresponding covalent incorporation, nanogel collapse does not influence the alginate matrix, and can leave voids in the walls of the macroporous structure, leading to a decrease in overall stiffness and elasticity.

The strategy of stiffening based on the collapse of NIPAM has been shown to result in a significant decrease in volume for non-macroporous structures.ⁱ However, for the cryogel composites presented herein, the collapse of the nanogels at elevated temperatures results in a contraction of the walls of the macroporous structure, which does not significantly influence the overall volume. Importantly, a significant decrease in volume associated with stiffening would not be desirable for embolization devices, as this could cause the material to lose contact with the blood vessel wall, leading to distal non-target embolization.

The stiffening effect observed in the 1% polymer loading macroporous composite was not observed in 1.5% polymer loading composites of with the same alginate-to-NIPAM ratio, either with or without functionalization of the NNGs. This suggests that the ideal alginate-to-NIPAM ratio may differ based on polymer loading percentage.

The size of the nanogels was also shown to be easily adjusted based on the concentration of surfactant during polymerization (FIG. 18).

### Example 4: Cryogel Synthesis Protocol I

This example details an exemplary synthesis protocol, mold designs, and results involving the creation of a patterned, anisotropic cryogel which can fold in a pre-designed fashion. Such cryogels can be folded into a catheter. By unfolding inside of a blood vessel in addition to swelling, these cryogels can be used to block significantly larger vessels than their unpatterned counterparts.

### Outer Layer (1.5% Methacrylated Alginate, 0% PCL Nanofibers)

1. Measure out 1.5% methacrylated alginate (MA-alginate) by weight (3.6 mg).
2. Disperse the MA-alginate and fibers into the 140 μL degassed and deionized water until no noticeable aggregates of MA-alginate and fibers are not visible.
3. Secure the tubes with the MA-alginate-fiber solution on a shaker and shake vigorously for an hour to ensure complete dispersion.
4. Prepare the 0.2% weight by volume of the initiators ammonium persulfate and TEMED and keep in ice. This can be achieved by dissolving 9.6 mg of APS in 1 mL of −5° C. water and 12.4 μL of TEMED in 987.6 μL of −5° C. water. Because the reaction and reagents are light-sensitive, wrap the individual reagents with aluminum foil and store in a dark room. Precool the cryogel molds in −20° C. Put the molds over ice to slow down the rate of temperature increase while pipetting cryogel solution into the wells.
5. Add APS solution to the MA-alginate-fiber solution. Mix well quickly. Immediately add TEMED and mix well. Cut two mm from the tip of a pipette tip so the initiated solution will not solidify in the pipette tip. After filling the wells, put the molds back in −20° C. After 4 hours, let the cryogels thaw before removing the inner ring and synthesizing the inner layer.

### Inner Layer (0.5% Methacrylated MA-Alginate, 0% PCL Nanofibers):

6. Measure out 0.5% methacrylated MA-alginate by weight (1.2 mg). Disperse the MA-alginate and fibers into the 140 μL degassed and deionized water until noticeable aggregates of MA-alginate and fibers are not visible. Secure the tubes with the MA-alginate-fiber solution on a shaker and shake vigorously for an hour to ensure complete dispersion.
7. Prepare the 0.2% weight by volume of the initiators ammonium persulfate and TEMED and keep in ice. This can be achieved by dissolving 9.6 mg of APS in 1 mL of −5 degrees Celsius water and 12.4 μL of TEMED in 984.6 μL of −5° C. water. Because the reaction and reagents are light-sensitive, wrap the individual reagents with aluminum foil and store in a dark room.
8. Precool the cryogel molds at −20° C. Put the molds over ice to slow down the rate of temperature increase while pipetting cryogel solution into the wells. Add APS solution to the MA-alginate-fiber solution. Mix well quickly. Immediately add TEMED and mix well.
9. Cut two mm from the tip of a pipette tip so the initiated solution will not solidify in the pipette tip. After filling the wells of the inner layer, put the molds back in −20° C. After 24 hours, store the concentric cryogel in distilled water at room temperature.

To compare the interface strength of the 2 layers, repeat the process, but synthesize the inner layer after letting the outer layer initiate for 24 hours, rather than 4 hours. Conduct injection tests to quantify strength of anisotropic cryogel.

Using the doubly wedged mold, we made a patterned cryogel with 2 different MA-alginate compositions. The stiffer cryogel layer was synthesized first with this mold, with the two wedges creating gaps which were later filled by the softer cryogel, as in the synthesis protocol described above. The 2 bands of softer cryogel serve as flexible hinges for the 3 areas of higher MA-alginate by weight. The images sufficiently prove that 1.5 wt % MA-alginate cryogel and 0.5 wt % MA-alginate cryogel can be patterned to display anisotropic folding, while the interface strength is enough to prevent the different cryogel from fracturing. See FIGS. 24A-D.

### Example 5: Cryogel Synthesis Protocol II

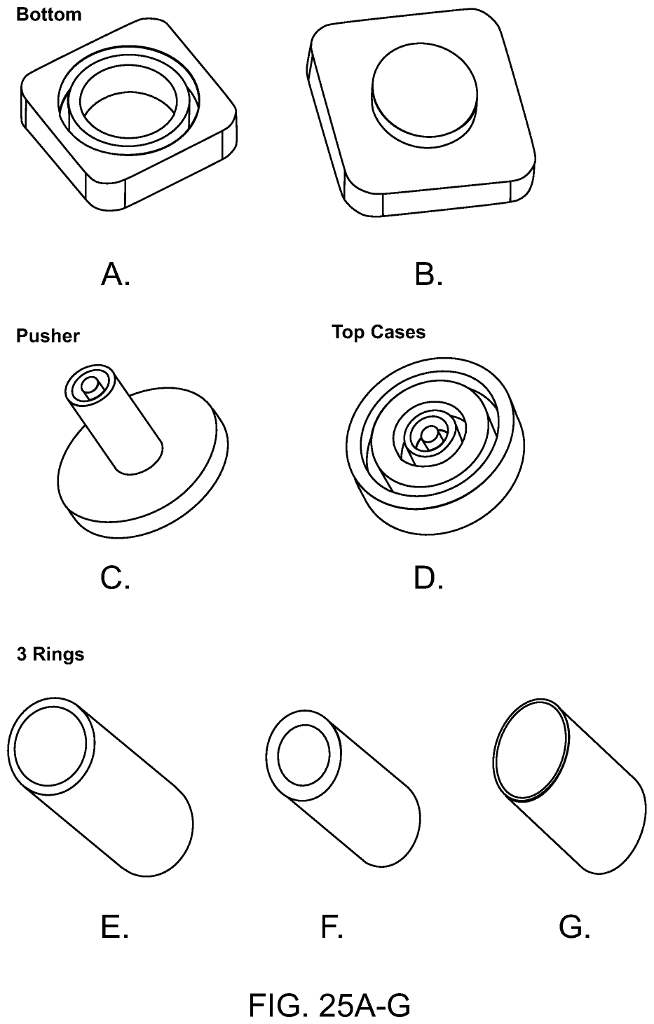

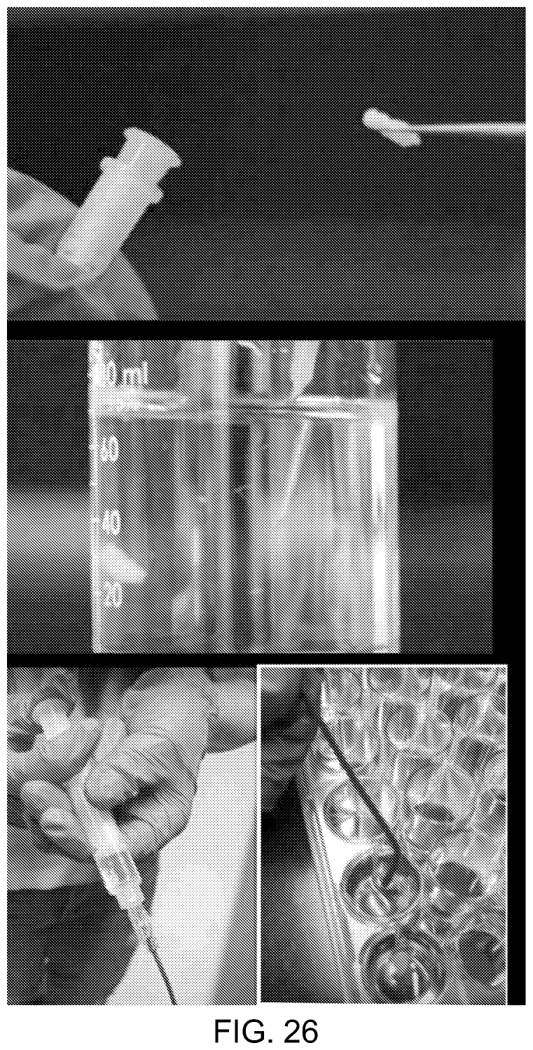

We created a cryogel with concentric rings of different stiffness. The stiff and soft layers were both 1% MA-alginate, but had 1.5% fibers and 0.5% fibers respectively, and were synthesized using the same protocol described above. A diagram of the molds is shown in FIGS. 25A-G. This cryogel was synthesized and extracted from the mold successfully. It was able to expand to full size upon hydration within 1 second, and no interface shearing was observed. These results show that a similar patterning is achievable in a more complex shape, with interfacially-bound nanofibers.

### Example 6: Injectability and Expansion Ratio Validation

This example describes the injectability protocol and the in vitro expansion ratio validation process for our unpatterned cryogel composite. Results of this experiment showed that the present gels are sufficiently mechanically robust to be injected through 16G catheters while remaining intact. The in vitro expansion ratio was found to be at least 6.7 fold.

Method 1. Synthesize 1% methacrylated alginate, 1% PCL nanofiber cryogels according to the cryogel synthesis protocol (diameter=8 mm). This formulation was chosen based on its superior mechanical and swelling properties.
2. Immerse the cryogel in 50 mM or 100 mM CaCl₂ for either 30 minutes or overnight. This provides extra ionic crosslinking to stiffen the gel for injection.
3. Fold and load the half/quarter cryogels in a semi-dehydrated state into a 16G needle (inner diameter=1.194 mm). At least 6 samples were prepared for each condition.
4. Attach the needle to a syringe/catheter filled with water and push out the device.
5. Score the injectability of the cryogel according to the following standard: 0—doesn't go through the needle, 1—gel broke into scattered pieces, 2—gel broke into halves, 3—gel was a little broken, 4—fully intact.
6. Repeat the procedure for 1.5% methacrylated alginate, 1% PCL nanofiber cryogels to test for the optimal concentration.

### Results:

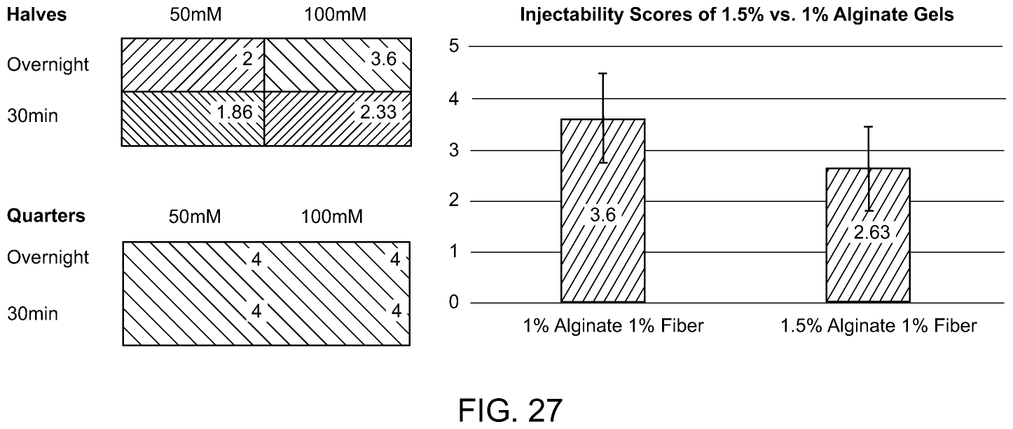

The injectability scores of 1% alginate 1% fiber half/quarter gels immersed in 50 mM or 100 mM CaCl₂ for 30 minutes or overnight is shown in FIG. 27. All quarter gels (4 mm in diameter) were able to come out of the needle intact. For half gels (8 mm in diameter), 100 mM CaCl₂ immersed overnight was shown to have the highest injectability score. The 1.5% alginate 1% fiber half gels immersed in 100 mM CaCl₂ for overnight (best condition) had an injectability score of 2.63. Therefore, the 1% alginate 1% fiber half gels immersed in 100 mM CaCl₂ overnight was taken as our best candidate. The cryogel was able to expand at least 6.7 fold from the 1.194 mm diameter catheter to its original size (8 mm).

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. An embolization composition comprising:
(a) a crosslinked hydrogel material; and
(b) a fiber material;
wherein:
the composition comprises a plurality of macropores; and
the hydrogel material and fiber material are bonded by covalent and/or non-covalent bonds.

2. The composition of claim 1 wherein the macropores have a pore size of at least 10 microns.

3. The composition of claim 1 wherein the macropores have a pore size of at least 50 microns.

4. The composition of claim 1 wherein the hydrogel material and fiber material are bonded by one or more electron sharing bonds.

5. The composition of claim 4 wherein the electron sharing bonds are one or more of an ionic bond, hydrogen bond and covalent bond.

6. The composition of claim 1 wherein the hydrogel material and the fiber material are covalently linked.

7. The composition of claim 6 wherein the covalent linkage comprises a reacted acrylate.

8. The composition of claim 6 wherein the covalent linkage comprises a reacted acrylamide, reacted maleimide and/or a reacted vinyl ether moiety.

9. The composition of claim 1 wherein the hydrogel material and the fiber material are associated by ionic bond interactions.

10. The composition of claim 1 wherein the hydrogel material and/or the fiber material comprise functional groups that can form ionic bonds between the hydrogel and fiber material.

11. The composition of claim 1 wherein the hydrogel material comprises polar moieties.

12. The composition of claim 11 wherein the polar moieties comprise hydroxy, carboxy, cyano and/or nitro groups.

13. The composition of claim 1 wherein the hydrogel material and fiber material are a cryogel composite.

14. The composition of claim 1 wherein the hydrogel material and fiber material are not significantly covalently linked.

15. The composition of claim 1 any one of claims 1 through 14 wherein the hydrogel material comprises reactive groups distinct from one or more polar moieties.

16. The composition of claim 15 wherein distinct reactive groups of the hydrogel material comprises acrylate groups.

17-35. (canceled)

36. An embolization composition comprising regions of differing stiffness; or
An embolization composition that comprises one or more therapeutic agents in differing amounts in distinct composition regions.

37-46. (canceled)

47. A method for embolizing a blood vessel of a subject, comprising:
delivering via a catheter into the blood vessel a composition of claim 1.

48. (canceled)

49. A method for treating a subject suffering from cancer, comprising:
administering to the subject a composition of claim 1.

50-54. (canceled)

55. A method for making an embolization composition, comprising:
admixing a hydrogel material and a fiber material;
forming a cryogel composite.

56. (canceled)
